(19) United States
(12) Patent Application Publication
(10) Pub. No.: US 2021/0400933 A1
(43) Pub. Date: Dec. 30, 2021

(54) GENETICALLY MODIFIED ANIMAL WITH CANINE OR CHIMERIC PD-1

(71) Applicants: Biocytogen Pharmaceuticals (Beijing) Co., Ltd., Beijing (CN); Biocytogen Jiangsu Co., Ltd., Jiangsu (CN)

(72) Inventors: Yuelei Shen, Beijing (CN); Yang Bai, Beijing (CN); Rui Huang, Beijing (CN); Xiaofei Zhou, Beijing (CN); Chengzhang Shang, Beijing (CN); Meiling Zhang, Beijing (CN); Jiawei Yao, Beijing (CN); Chaoshe Guo, Beijing (CN); Yanan Guo, Beijing (CN)

(21) Appl. No.: 17/295,256

(22) PCT Filed: Nov. 19, 2019
(86) PCT No.: PCT/CN2019/119492

§ 371 Date: May 19, 2021

(30) Foreign Application Priority Data
Nov. 19, 2018 (CN) 201811381674.5

(51) Int. Cl. A01K 67/027; C12N 15/85; C07K 14/705

(52) U.S. Cl. CPC A01K 67/0275; C12N 15/8509; A01K 2267/0331; A01K 2217/072; A01K 2227/105; C07K 14/70521

(57) ABSTRACT

The present disclosure relates to genetically modified animals that express a canine or chimeric (e.g., caninized) programmed cell death protein 1 (PD-1), and methods of use thereof.

### CLAIM OF PRIORITY

This application claims the benefit of Chinese Patent Application App. No. 201811381674.5, filed on Nov. 19, 2018. The entire contents of the foregoing are incorporated herein by reference.

### TECHNICAL FIELD

This disclosure relates to genetically modified animal expressing canine or chimeric (e.g., caninized) PD-1, and methods of use thereof.

### BACKGROUND

The immune system has developed multiple mechanisms to prevent deleterious activation of immune cells. One such mechanism is the intricate balance between positive and negative costimulatory signals delivered to immune cells. Targeting the stimulatory or inhibitory pathways for the immune system is considered to be a potential approach for the treatment of various diseases, e.g., cancers and autoimmune diseases.

The traditional drug research and development for these stimulatory or inhibitory receptors typically use in vitro screening approaches. However, these screening approaches cannot provide the body environment (such as tumor microenvironment, stromal cells, extracellular matrix components and immune cell interaction, etc.), resulting in a higher rate of failure in drug development. In addition, in view of the differences between humans and animals, the test results obtained from the use of conventional experimental animals for in vivo pharmacological test may not reflect the real disease state and the interaction at the targeting sites, resulting in that the results in many clinical trials are significantly different from the animal experimental results. Therefore, the development of animal models that are suitable for antibody screening and evaluation will significantly improve the efficiency of new drug development and reduce the cost for drug research and development.

### SUMMARY

This disclosure is related to an animal model (e.g., non-human animal) with canine PD-1 or chimeric PD-1. The animal model can express canine PD-1 or chimeric PD-1 (e.g., caninized PD-1) protein in its body. It can be used in the studies on the function of PD-1 gene, and can be used in the screening and evaluation of anti-canine PD-1 antibodies. In addition, the animal models prepared by the methods described herein can be used in drug screening, pharmacodynamics studies, treatments for immune-related diseases (e.g., autoimmune disease), and cancer therapy for PD-1 target sites; they can also be used to facilitate the development and design of new drugs, and save time and cost. In summary, this disclosure provides a powerful tool for studying the function of PD-1 protein and a platform for screening cancer drugs.

In one aspect, the disclosure relates to a genetically-modified, non-human, non-canine animal whose genome comprises at least one chromosome comprising a sequence encoding a canine or chimeric PD-1.

In some embodiments, the sequence encoding the canine or chimeric PD-1 is operably linked to an endogenous regulatory element at the endogenous PD-1 gene locus in the at least one chromosome.

In some embodiments, the sequence encoding a canine or chimeric PD-1 comprises a sequence encoding an amino acid sequence that is at least 70%, 75%, 80%, 85%, 90%, 95%, 99%, or 100% identical to canine PD-1 (NP 001301026.1 (SEQ ID NO: 4)). In some embodiments, the sequence encoding a canine or chimeric PD-1 comprises a sequence encoding an amino acid sequence that is at least 70%, 75%, 80%, 85%, 90%, 95%, 99%, or 100% identical to SEQ ID NO: 8.

In some embodiments, the animal is a mammal, e.g., a monkey, a rodent or a mouse. In some embodiments, the animal is a mouse or a rat.

In some embodiments, the animal does not express endogenous PD-1. In some embodiments, the animal has one or more cells expressing canine or chimeric PD-1.

In some embodiments, the animal has one or more cells expressing canine or chimeric PD-1, and canine PD-L1 or canine PD-L2 can bind to the expressed canine or chimeric PD-1. In some embodiments, the animal has one or more cells expressing canine or chimeric PD-1, and endogenous PD-L1 or endogenous PD-L2 can bind to the expressed canine or chimeric PD-1.

In one aspect, the disclosure relates to a genetically-modified, non-human, non-canine animal, wherein the genome of the animal comprises a replacement of a sequence encoding a region of endogenous PD-1 with a sequence encoding a canine PD-1 or a chimeric PD-1 at an endogenous PD-1 gene locus.

In some embodiments, the sequence encoding the canine PD-1 or the chimeric PD-1 is operably linked to an endogenous regulatory element at the endogenous PD-1 locus, and one or more cells of the animal express the canine PD-1 or the chimeric PD-1. In some embodiments, the animal does not express endogenous PD-1.

In some embodiments, the replaced locus is located after start codon at the endogenous PD-1 locus.

In some embodiments, the animal has one or more cells expressing a chimeric PD-1 having an extracellular region, a transmembrane region, and a cytoplasmic region. In some embodiments, the extracellular region comprises a sequence that is at least 50%, 60%, 70%, 80%, 90%, 95%, or 99% identical to the extracellular region of canine PD-1.

In some embodiments, the extracellular region of the chimeric PD-1 has a sequence that has at least 10, 20, 30, 40, 50, 60, 70, 80, 90, or 100 contiguous amino acids that are identical to a contiguous sequence present in the extracellular region of canine PD-1.

In some embodiments, the animal is a mouse, and the replaced region is in exon 2 of the endogenous mouse PD-1 gene. In some embodiments, the animal is heterozygous with respect to the replacement at the endogenous PD-1 gene locus.

In some embodiments, the animal is homozygous with respect to the replacement at the endogenous PD-1 gene locus.

In one aspect, the disclosure relates to a method for making a genetically-modified, non-human, non-canine animal, comprising: replacing in at least one cell of the animal, at an endogenous PD-1 gene locus, a sequence encoding a region of an endogenous PD-1 with a sequence comprising at least one exon of canine PD-1 gene or at least one chimeric exon (e.g., canine/mouse chimeric exon).

In some embodiments, the sequence comprising at least one exon of canine PD-1 gene comprises exon 1, exon 2, exon 3, exon 4, and/or exon 5, or a part thereof, of a canine PD-1 gene.

In some embodiments, the sequence comprising at least one exon of canine PD-1 gene comprises exon 1, exon 2, and/or exon 3, or a part thereof, of a canine PD-1 gene.

In some embodiments, the sequence comprising at least a sequence encoding at least amino acids 31-141 of SEQ ID NO: 4.

In some embodiments, the animal is a mouse, and the endogenous PD-1 locus is located at exon 1, exon 2, exon 3, exon 4, and/or exon 5 of the mouse PD-1 gene. In some embodiments, the region is located in exon 2 of the mouse PD-1 gene. In some embodiments, the entire exon 2 or part of exon 2 is replaced with canine PD-1.

In one aspect, the disclosure relates to a non-human animal comprising at least one cell comprising a nucleotide sequence encoding a chimeric PD-1 polypeptide. In some embodiments, the chimeric PD-1 polypeptide comprises at least 50 contiguous amino acid residues that are identical to the corresponding contiguous amino acid sequence of a canine PD-1. In some embodiments, the animal expresses the chimeric PD-1.

In some embodiments, the chimeric PD-1 polypeptide has at least 50 contiguous amino acid residues that are identical to the corresponding contiguous amino acid sequence of a canine PD-1 extracellular region.

In some embodiments, the chimeric PD-1 polypeptide comprises a sequence that is at least 90%, 95%, or 99% identical to amino acids 31-141 of SEQ ID NO: 4.

In some embodiments, the nucleotide sequence is operably linked to an endogenous PD-1 regulatory element of the animal.

In some embodiments, the chimeric PD-1 polypeptide comprises an endogenous PD-1 transmembrane region and/or an endogenous PD-1 cytoplasmic region.

In some embodiments, the nucleotide sequence is integrated to an endogenous PD-1 gene locus of the animal.

In some embodiments, the chimeric PD-1 has at least one mouse PD-1 activity and/or at least one canine PD-1 activity.

In one aspect, the disclosure relates to a method of making a genetically-modified mouse cell that expresses a canine PD-1 or a chimeric PD-1, the method comprising: replacing at an endogenous mouse PD-1 gene locus, a nucleotide sequence encoding a region of mouse PD-1 with a nucleotide sequence encoding a canine PD-1 or a chimeric PD-1, thereby generating a genetically-modified mouse cell that includes a nucleotide sequence that encodes the canine PD-1 or the chimeric PD-1. In some embodiments, the mouse cell expresses the canine PD-1 or the chimeric PD-1.

In some embodiments, the chimeric PD-1 comprises: an extracellular region of canine PD-1; and a transmembrane and/or a cytoplasmic region of mouse PD-1.

In some embodiments, the nucleotide sequence encoding the canine PD-1 or the chimeric PD-1 is operably linked to an endogenous PD-1 regulatory region, e.g., a promoter.

In some embodiments, the animal further comprises a sequence encoding an additional canine or chimeric protein. In some embodiments, the additional canine or chimeric protein is cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD3, CD27, CD28, CD40, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), SIRPA (Signal Regulatory Protein Alpha), or TNF Receptor Superfamily Member 4 (OX40).

In some embodiments, the animal or mouse further comprises a sequence encoding an additional canine or chimeric protein. In some embodiments, the additional canine or chimeric protein is CTLA-4, LAG-3, BTLA, PD-L1, CD3, CD3e, CD27, CD28, CD40, CD47, CD137, CD154, SIPRA, TIGIT, TIM-3, GITR, or OX40.

In one aspect, the disclosure relates to a method of determining effectiveness of an anti-PD-1 antibody for the treatment of cancer. The method includes the steps of administering the anti-PD-1 antibody to the animal as described herein, wherein the animal has a tumor; and determining the inhibitory effects of the anti-PD-1 antibody to the tumor.

In some embodiments, the tumor comprises one or more cells that express a PD-1 ligand. In some embodiments, the tumor comprises one or more cancer cells that are injected into the animal. In some embodiments, determining the inhibitory effects of the anti-PD-1 antibody to the tumor comprises measuring the tumor volume in the animal.

In some embodiments, the tumor cells are melanoma cells, pancreatic carcinoma cells, mesothelioma cells, or solid tumor cells.

In one aspect, the disclosure relates to a method of determining effectiveness of an anti-PD-1 antibody and an additional therapeutic agent for the treatment of a tumor, comprising administering the anti-PD-1 antibody and the additional therapeutic agent to the animal as described herein, wherein the animal has a tumor; and determining the inhibitory effects on the tumor.

In some embodiments, the animal further comprises a sequence encoding a canine or chimeric CTLA4. In some embodiments, the animal further comprises a sequence encoding a canine or chimeric programmed death-ligand 1 (PD-L1).

In some embodiments, the additional therapeutic agent is an anti-PD-L1 antibody or an anti-CTLA4 antibody. In some embodiments, the tumor comprises one or more tumor cells that express PD-L1 or PD-L2.

In some embodiments, the tumor is caused by injection of one or more cancer cells into the animal. In some embodiments, determining the inhibitory effects of the treatment involves measuring the tumor volume in the animal.

In some embodiments, the animal has melanoma, pancreatic carcinoma, mesothelioma, hematological malignancies (e.g., Non-Hodgkin's lymphoma, lymphoma, chronic lymphocytic leukemia), or solid tumors.

In one aspect, the disclosure relates to a protein comprising an amino acid sequence, wherein the amino acid sequence is one of the following:

(a) an amino acid sequence set forth in SEQ ID NO: 8;

(b) an amino acid sequence that is at least 90% identical to SEQ ID NO: 8;

(c) an amino acid sequence that is at least 91%, 92%, 93%, 94%, 95%, 96%, 97%, 98%, or 99% identical to SEQ ID NO: 8;

(d) an amino acid sequence that is different from the amino acid sequence set forth in SEQ ID NO: 8 by no more than 10, 9, 8, 7, 6, 5, 4, 3, 2 or 1 amino acid; and

(e) an amino acid sequence that comprises a substitution, a deletion and/or insertion of one, two, three, four, five or more amino acids to the amino acid sequence set forth in SEQ ID NO: 8.

In one aspect, the disclosure relates to a nucleic acid comprising a nucleotide sequence, wherein the nucleotide sequence is one of the following:

(a) a sequence that encodes the protein as described herein;

(b) SEQ ID NO: 5, SEQ ID NO: 6, or SEQ ID NO: 7;

(c) a sequence that is at least 90% identical to SEQ ID NO: 5, SEQ ID NO: 6, or SEQ ID NO: 7; and

(d) a sequence that is at least 91%, 92%, 93%, 94%, 95%, 96%, 97%, 98%, or 99% identical to SEQ ID NO: 5, SEQ ID NO: 6, or SEQ ID NO: 7.

In one aspect, the disclosure relates to a cell comprising the protein as described herein and/or the nucleic acid as described herein. In one aspect, the disclosure relates to an animal comprising the protein as described herein and/or the nucleic acid as described herein.

In one aspect, the disclosure relates to a method of determining effectiveness of an anti-PD-L1 antibody for the treatment of cancer, comprising: administering the anti-PD-L1 antibody to the animal as described herein, wherein the animal has a tumor; and determining the inhibitory effects of the anti-PD-L1 antibody to the tumor.

In some embodiments, the tumor comprises one or more cells that express PD-L1. In some embodiments, the tumor comprises one or more cancer cells that are injected into the animal. In some embodiments, determining the inhibitory effects of the anti-PD-L1 antibody to the tumor comprises measuring the tumor volume in the animal.

In one aspect, the disclosure provides a cell comprising the protein and/or the nucleic acid as described herein.

In some embodiments, the tumor comprises one or more cancer cells that are injected into the animal. In some embodiments, determining the inhibitory effects of the anti-PD-1 antibody to the tumor involves measuring the tumor volume in the animal. In some embodiments, the tumor cells are melanoma cells (e.g., advanced melanoma cells), non-small cell lung carcinoma (NSCLC) cells, small cell lung cancer (SCLC) cells, bladder cancer cells, non-Hodgkin lymphoma cells, and/or prostate cancer cells (e.g., metastatic hormone-refractory prostate cancer). In some embodiments, the tumor cells are hepatocellular, ovarian, colon, or cervical tumor cells. In some embodiments, the tumor cells are breast cancer cells, ovarian cancer cells, and/or solid tumor cells. In some embodiments, the tumor cells are lymphoma cells, colorectal cancer cells, or oropharyngeal cancer cells. In some embodiments, the animal has metastatic solid tumors, NSCLC, melanoma, lymphoma (e.g., non-Hodgkin lymphoma), colorectal cancer, or multiple myeloma. In some embodiments, the animal has melanoma, pancreatic carcinoma, mesothelioma, hematological malignancies (e.g., Non-Hodgkin's lymphoma, lymphoma, chronic lymphocytic leukemia), or solid tumors.

In one aspect, the disclosure relates to methods of determining effectiveness of an anti-PD-1 antibody for the treatment of various immune-related disorders, e.g., autoimmune diseases.

In another aspect, the disclosure also provides a genetically-modified animal whose genome comprise a disruption in the animal's endogenous PD-1 gene, wherein the disruption of the endogenous PD-1 gene comprises deletion of exon 1, exon 2, exon 3, exon 4, and/or exon 5, or part thereof of the endogenous PD-1 gene.

In some embodiments, the disruption of the endogenous PD-1 gene comprises deletion of one or more exons or part of exons selected from the group consisting of exon 1, exon 2, exon 3, exon 4, and exon 5 of the endogenous PD-1 gene.

In some embodiments, the disruption of the endogenous PD-1 gene further comprises deletion of one or more introns or part of introns selected from the group consisting of intron 1, intron 2, intron 3, and intron 4 of the endogenous PD-1 gene.

In some embodiments, wherein the deletion can comprise deleting at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, 300, 350, 400, 450, 500, 550, 600, 650, or more nucleotides.

In some embodiments, the disruption of the endogenous PD-1 gene comprises the deletion of at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 10, 220, 230, 240, 250, 260, 270, 280, 290, or 300 nucleotides of exon 1, exon 2, exon 3, exon 4, and/or exon 5 (e.g., deletion of at least 300 nucleotides of exon 2).

In some embodiments, the mice described in the present disclosure can be mated with the mice containing other canine or chimeric genes (e.g., chimeric PD-L1, chimeric PD-L2, chimeric CTLA-4, or other immunomodulatory factors), so as to obtain a mouse expressing two or more canine or chimeric proteins. The mice can also, e.g., be used for screening antibodies in the case of a combined use of drugs, as well as evaluating the efficacy of the combination therapy.

In another aspect, the disclosure further provides methods of determining toxicity of an agent (e.g., a PD-1 antagonist or agonist). The methods involve administering the agent to the animal as described herein; and determining weight change of the animal. In some embodiments, the method further involve performing a blood test (e.g., determining red blood cell count).

In one aspect, the disclosure relates to a targeting vector, including a) a DNA fragment homologous to the 5′ end of a region to be altered (5′ arm), which is selected from the PD-1 gene genomic DNAs in the length of 100 to 10,000 nucleotides; b) a desired/donor DNA sequence encoding a donor region or a protein (e.g., a canine PD-1 or a chimeric PD-1); and c) a second DNA fragment homologous to the 3′ end of the region to be altered (3′ arm), which is selected from the PD-1 gene genomic DNAs in the length of 100 to 10,000 nucleotides.

In some embodiments, a) the DNA fragment homologous to the 5′ end of a region to be altered (5′ arm/receptor) is selected from the nucleotide sequences that have at least 90% homology to the NCBI accession number NC_000067.6; c) the DNA fragment homologous to the 3′ end of the region to be altered (3′ arm/receptor) is selected from the nucleotide sequences that have at least 90% homology to the NCBI accession number NC_000067.6.

In some embodiments, a) the DNA fragment homologous to the 5′ end of a region to be altered (5′ arm/receptor) is selected from the nucleotides from the position 94041502 to the position 94043271 of the NCBI accession number NC_000067.6; c) the DNA fragment homologous to the 3′ end of the region to be altered (3′ arm/receptor) is selected from the nucleotides from the position 94039436 to the position 94041168 of the NCBI accession number NC_000067.6.

In some embodiments, a length of the selected genomic nucleotide sequence is more than 300 bp, 400 bp, 500 bp, 1 kb, 2 kb, 3 kb, 3.5 kb, 4 kb, 4.5 kb, 5 kb, 5.5 kb, or 6 kb. In some embodiments, the region to be altered is exon 1, exon 2, exon 3, exon 4, and/or exon 5 (e.g., exon 2) of mouse PD-1 gene.

In some embodiments, the sequence of the 5′ arm is shown in SEQ ID NO: 9. In some embodiments, the sequence of the 3′ arm is shown in SEQ ID NO: 10.

In some embodiments, the targeting vector further includes a selectable gene marker.

In some embodiments, the target region is derived from a dog. In some embodiments, the target region is a part or entirety of the nucleotide sequence of a canine PD-1 or a chimeric PD-1. In some embodiments, the nucleotide sequence is shown as one or more of exon 1, exon 2, exon 3, exon 4, and exon 5 (e.g., exon 2) of the canine PD-1.

In some embodiments, the nucleotide sequence of the canine PD-1 encodes the canine PD-1 protein NP 001301026.1 (SEQ ID NO: 4). In some embodiments, the canine PD-1 gene fragment (SEQ ID NO: 11) has a mutation relative to nucleic acid 51611212-51611544 of NCBI Accession No. NC_006607.3, that is, a T at position 203 is mutated to C.

The disclosure also relates to a cell including the targeting vector as described herein.

The disclosure also relates to a method for establishing a genetically-modified animal expressing two canine or chimeric (e.g., caninized) genes. The method includes the steps of

(a) using the method for establishing a PD-1 gene caninized animal model to obtain a PD-1 gene genetically modified caninized mouse;

(b) mating the PD-1 gene genetically modified caninized mouse obtained in step (a) with another caninized mouse, and then screening to obtain a double caninized mouse model.

In some embodiments, in step (b), the PD-1 gene genetically modified caninized mouse obtained in step (a) is mated with a PD-L1 caninized mouse to obtain a PD-1 and PD-L1 double caninized mouse model.

The disclosure also relates to a mammal generated through the methods as described herein.

In some embodiments, the genome thereof contains canine gene(s).

In some embodiments, the mammal is a rodent. In some embodiments, the rodent is a mouse or a rat.

In some embodiments, the mammal expresses a protein encoded by a canine PD-1 gene or a chimeric PD-1 gene.

The disclosure also relates to an offspring of the mammal.

In another aspect, the disclosure relates to a tumor bearing mammal model, wherein the mammal model is obtained through the methods as described herein.

The disclosure also relates to a cell (e.g., stem cell or embryonic stem cell) or cell line, or a primary cell culture thereof derived from the non-human mammal or an offspring thereof, or the tumor bearing non-human mammal. The disclosure further relates to the tissue, organ or a culture thereof derived from the non-human mammal or an offspring thereof, or the tumor bearing non-human mammal.

In another aspect, the disclosure relates to a tumor tissue derived from the non-human mammal or an offspring thereof when it bears a tumor, or the tumor bearing non-human mammal.

The disclosure further relates to a PD-1 genomic DNA sequence of a caninized mouse, a DNA sequence obtained by a reverse transcription of the mRNA obtained by transcription thereof is consistent with or complementary to the DNA sequence; a construct expressing the amino acid sequence thereof; a cell comprising the construct thereof; a tissue comprising the cell thereof.

The disclosure further relates to the use of the mammal or an offspring thereof, or the tumor bearing mammal, the animal model generated through the methods as described herein, in the screening, verifying, evaluating or studying the PD-1 gene function, anti-canine PD-1 antibodies, anti-canine PD-L1 antibodies, the drugs or efficacies for canine PD-1 or PD-L1 targeting sites, and the drugs for immune-related diseases and antitumor drugs.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram showing the mouse PD-1 gene and the canine PD-1 gene locus.

FIG. 2 is a schematic diagram showing a caninized mouse PD-1 gene locus.

FIG. 3 is a schematic diagram showing a gene targeting strategy for embryonic stem cells for a sequence encoding caninized mouse PD-1 amino acid sequence.

FIG. 4A shows the restriction enzymes digestion results of the targeting plasmid pClon-4G-DPD-1 by two sets of restriction enzymes. Ck indicates undigested plasmids, which were used as a control. M is the Marker. No. 1-10 are plasmid numbers.

FIG. 4B shows DNA ladder for the Marker.

FIG. 5 shows the restriction enzymes digestion results of the targeting plasmid pClon-4G-DPD-1 by restriction enzymes KpnI and BamHI. Ck indicates undigested plasmids, which were used as a control. M is the Marker. No. 2, 3, 4, 5, 6, 9, and 10 are plasmid numbers.

FIG. 6 is a graph showing activity testing results for sgRNA1-sgRNA8 (Con is a negative control; PC is a positive control; Blank is a blank control).

FIG. 7 shows PCR identification results of samples collected from tails of F0 generation mice. WT is wild-type. H₂O is a blank control and M is the Marker. F0-1 to F0-10 are labels for F0 generation mice.

FIG. 8 shows PCR identification results of samples collected from tails of F0 generation mice. WT is wild-type. H₂O is a blank control and M is the Marker.

FIG. 9 shows PCR identification results of samples collected from PD-1 gene knockout mice. WT is wild-type. H₂O is a blank control and M is the Marker. KO-1 to KO-3 are labels for mice.

FIG. 10. The average weight of the different groups of caninized PD-1 homozygous mice that were injected with mouse colon cancer cells MC38, and were treated with 3 different anti-canine PD-1 antibodies (Ab1, Ab2 and Ab3) at a dosage of 10 mg/kg.

FIG. 11. The percentage change of average weight of the different groups of caninized PD-1 homozygous mice that were injected with mouse colon cancer cells MC38, and were treated with 3 different anti-canine PD-1 antibodies (Ab1, Ab2 and Ab3) at a dosage of 10 mg/kg.

FIG. 12. The average tumor volume in the different groups of caninized PD-1 homozygous mice that were injected with mouse colon cancer cells MC38, and were treated with 3 different anti-canine PD-1 antibodies (Ab1, Ab2 and Ab3) at a dosage of 10 mg/kg.

FIG. 13. The average weight of the different groups of caninized PD-1 homozygous mice that were injected with mouse colon cancer cells MC38, and were treated with an anti-canine PD-1 antibody at different dosages (10 mg/kg, 3 mg/kg or 0.3 mg/kg).

FIG. 14. The percentage change of average weight of the different groups of caninized PD-1 homozygous mice that were injected with mouse colon cancer cells MC38, and were treated with an anti-canine PD-1 antibody at different dosages (10 mg/kg, 3 mg/kg or 0.3 mg/kg).

FIG. 15. The average tumor volume in the different groups of caninized PD-1 homozygous mice that were injected with mouse colon cancer cells MC38, and were treated with an anti-canine PD-1 antibody at different dosages (10 mg/kg, 3 mg/kg or 0.3 mg/kg).

FIG. 16 shows the alignment between mouse PD-1 amino acid sequence (NP_032824.1; SEQ ID NO: 2) and canine PD-1 amino acid sequence (NP_001301026.1; SEQ ID NO: 4).

### DETAILED DESCRIPTION

This disclosure relates to transgenic non-human animal with canine or chimeric (e.g., caninized) PD-1 (Programmed Cell Death Protein 1; also known as CD279), and methods of use thereof.

The immune system can differentiate between normal cells in the body and those it sees as “foreign,” which allows the immune system to attack the foreign cells while leaving the normal cells alone. This mechanism sometimes involves proteins called immune checkpoints. Immune checkpoints are molecules in the immune system that either turn up a signal (co-stimulatory molecules) or turn down a signal.

Checkpoint inhibitors can prevent the immune system from attacking normal tissue and thereby preventing autoimmune diseases. Many tumor cells also express checkpoint inhibitors. These tumor cells escape immune surveillance by co-opting certain immune-checkpoint pathways, particularly in T cells that are specific for tumor antigens (Creelan, Benjamin C. “Update on immune checkpoint inhibitors in lung cancer.” Cancer Control 21.1 (2014): 80-89). Because many immune checkpoints are initiated by ligand-receptor interactions, they can be readily blocked by antibodies against the ligands and/or their receptors.

Experimental animal models are an indispensable research tool for studying the effects of these antibodies (e.g., PD-1 antibodies). Common experimental animals include mice, rats, guinea pigs, hamsters, rabbits, monkeys, pigs, fish and so on. However, there are many differences between genes and protein sequences from different species, and many proteins cannot bind to the animal's homologous proteins to produce biological activity. A large number of clinical studies are in urgent need of better animal models. This disclosure relates to transgenic non-human animal with canine or chimeric (e.g., caninized) PD-1 for testing anti-canine PD-1 antibodies.

Unless otherwise specified, the practice of the methods described herein can take advantage of the techniques of cell biology, cell culture, molecular biology, transgenic biology, microbiology, recombinant DNA and immunology. These techniques are explained in detail in the following literature, for examples: Molecular Cloning A Laboratory Manual, 2nd Ed., ed. By Sambrook, Fritsch and Maniatis (Cold Spring Harbor Laboratory Press: 1989); DNA Cloning, Volumes I and II (D. N. Glovered., 1985); Oligonucleotide Synthesis (M. J. Gaited., 1984); Mullis et al U. S. Pat. No. 4, 683, 195; Nucleic Acid Hybridization (B. D. Hames& S. J. Higginseds. 1984); Transcription And Translation (B. D. Hames& S. J. Higginseds. 1984); Culture Of Animal Cell (R. I. Freshney, Alan R. Liss, Inc., 1987); Immobilized Cells And Enzymes (IRL Press, 1986); B. Perbal, A Practical Guide To Molecular Cloning (1984), the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Caloseds., 1987, Cold Spring Harbor Laboratory); Immunochemical Methods In Cell And Molecular Biology (Mayer and Walker, eds., Academic Press, London, 1987); Hand book Of Experimental Immunology, Volumes V (D. M. Weir and C. C. Blackwell, eds., 1986); and Manipulating the Mouse Embryo, (Cold Spring Harbor Laboratory Press, Cold Spring Harbor, N.Y., 1986); each of which is incorporated herein by reference in its entirety.

### PD-1

PD-1 (Programmed cell death protein 1 or CD279) is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

PD-1 is mainly expressed on the surfaces of T cells and primary B cells; two ligands of PD-1 (PD-L1 and PD-L2) are widely expressed in antigen-presenting cells (APCs). The interaction of PD-1 with its ligands plays an important role in the negative regulation of the immune response. Inhibiting the binding between PD-1 and its ligand can make the tumor cells exposed to the killing effect of the immune system, and thus can reach the effect of killing tumor tissues and treating cancers.

PD-L1 is expressed on the neoplastic cells of many different cancers. By binding to PD-1 on T-cells leading to its inhibition, PD-L1 expression is a major mechanism by which tumor cells can evade immune attack. PD-L1 over-expression may conceptually be due to two mechanisms, intrinsic and adaptive. Intrinsic expression of PD-L1 on cancer cells is related to cellular/genetic aberrations in these neoplastic cells. Activation of cellular signaling including the AKT and STAT pathways results in increased PD-L1 expression. In primary mediastinal B-cell lymphomas, gene fusion of the MHC class II transactivator (CIITA) with PD-L1 or PD-L2 occurs, resulting in overexpression of these proteins. Amplification of chromosome 9p23-24, where PD-L1 and PD-L2 are located, leads to increased expression of both proteins in classical Hodgkin lymphoma. Adaptive mechanisms are related to induction of PD-L1 expression in the tumor microenvironment. PD-L1 can be induced on neoplastic cells in response to interferon γ. In microsatellite instability colon cancer, PD-L1 is mainly expressed on myeloid cells in the tumors, which then suppress cytotoxic T-cell function.

The use of PD-1 blockade to enhance anti-tumor immunity originated from observations in chronic infection models, where preventing PD-1 interactions reversed T-cell exhaustion. Similarly, blockade of PD-1 prevents T-cell PD-1/tumor cell PD-L1 or T-cell PD-1/tumor cell PD-L2 interaction, leading to restoration of T-cell mediated anti-tumor immunity.

A detailed description of PD-1, and the use of anti-PD-1 antibodies to treat cancers are described, e.g., in Topalian, Suzanne L., et al. “Safety, activity, and immune correlates of anti-PD-1 antibody in cancer.” New England Journal of Medicine 366.26 (2012): 2443-2454; Hirano, Fumiya, et al. “Blockade of B7-H1 and PD-1 by monoclonal antibodies potentiates cancer therapeutic immunity.” Cancer research 65.3 (2005): 1089-1096; Raedler, Lisa A. “Keytruda (pembrolizumab): first PD-1 inhibitor approved for previously treated unresectable or metastatic melanoma.” American health & drug benefits 8. Spec Feature (2015): 96; Kwok, Gerry, et al. “Pembrolizumab (Keytruda).” (2016): 2777-2789; US 20170247454; U.S. Pat. Nos. 9,834,606 B; and 8,728,474; each of which is incorporated by reference in its entirety.

In canine genomes, PD-1 gene (Gene ID: 486213) locus has five exons, exon 1, exon 2, exon 3, exon 4, and exon 5. The PD-1 protein also has an extracellular region, a transmembrane region, and a cytoplasmic region, and the signal peptide is located at the extracellular region of PD-1. The nucleotide sequence for canine PD-1 mRNA is NM 001314097.1 (SEQ ID NO: 3), and the amino acid sequence for canine PD-1 is NP NP_001301026.1 (SEQ ID NO: 4). The location for each exon and each region in canine PD-1 nucleotide sequence and amino acid sequence is listed below:

TABLE 1

| Canis lupus familiaris (dog) | | |
|---|---|---|
| PDCD1 | NM_001314097.1 | NP_001301026.1 |
| (approximate location) | 942 bp | 288aa |
| GENE ID: 486213 | SEQ ID NO: 3 | SEQ ID NO: 4 |
| Exon 1 | 1-122 | 1-25 |
| Exon 2 | 123-482 | 26-142 |
| Exon 3 | 483-644 | 146-199 |
| Exon 4 | 645-679 | 200-211 |
| Exon 5 | 680-942 | 212-288 |
| Signal peptide | 47-118 | 1-24 |
| Extracellular region | 119-550 | 25-168 |
| (excluding signal peptide | | |
| region) | | |
| Transmembrane region | 551-628 | 169-194 |
| Cytoplasmic region | 629-910 | 195-288 |
| Donor region in Example | 137-469 | 31-141 |

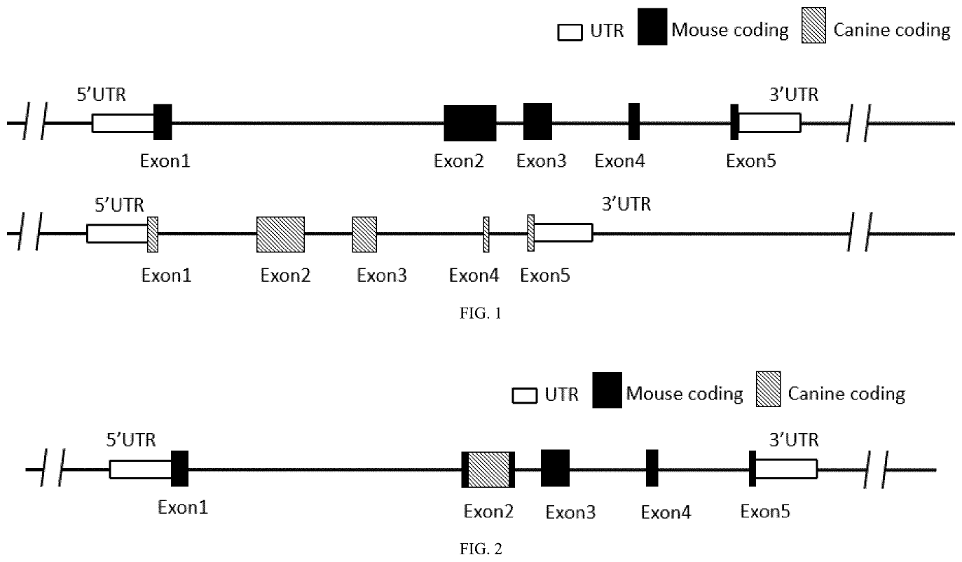

In mice, PD-1 gene locus has five exons, exon 1, exon 2, exon 3, exon 4, and exon 5 (FIG. 1). The mouse PD-1 protein also has an extracellular region, a transmembrane region, and a cytoplasmic region, and the signal peptide is located at the extracellular region of PD-1. The nucleotide sequence for mouse PD-1 mRNA is NM_008798.2 (SEQ ID NO: 1), the amino acid sequence for mouse PD-1 is NP_032824.1 (SEQ ID NO: 2). The location for each exon and each region in the mouse PD-1 nucleotide sequence and amino acid sequence is listed below:

TABLE 2

| Mouse Pdcd1 | NM_008798.2 | NP_032824.1 |
|---|---|---|
| (approximate location) | 1972 bp | 288aa |
| GENE ID: 18566 | SEQ ID NO: 1 | SEQ ID NO: 2 |
| Exon 1 | 1-139 | 1-25 |
| Exon 2 | 140-499 | 26-145 |
| Exon 3 | 500-661 | 146-199 |
| Exon 4 | 662-696 | 200-211 |
| Exon 5 | 697-1932 | 212-288 |
| Signal peptide | 64-123 | 1-20 |
| Extracellular region | 124-570 | 21-169 |
| (excluding signal peptide | | |
| region) | | |
| Transmembrane region | 571-633 | 170-190 |
| Cytoplasmic region | 634-927 | 191-288 |
| Replaced region in Example | 154-486 | 31-141 |

The mouse PD-1 gene (Gene ID: 18566) is located in Chromosome 1 of the mouse genome, which is located from 94038305-94052553, of NC_000067.6 (GRCm38.p4 (GCF_000001635.24)). The 5′-UTR is from 94052553 to 94052491, exon 1 is from 94052490 to 94052415, the first intron is from 94052414 to 94041516, exon 2 is from 94041515 to 94041156, the second intron is from 94041155 to 94040872, exon 3 is from 94040871 to 94040710, the third intron is from 94040709 to 94040127, exon 4 is from 94040126 to 94040092, the fourth intron is from 94040091 to 94039539, exon 5 is from 94039538 to 94039305, the 3′-UTR is from 94039304 to 94038305, based on transcript NM_008798.2. All relevant information for mouse PD-1 locus can be found in the NCBI website with Gene ID: 18566, which is incorporated by reference herein in its entirety.

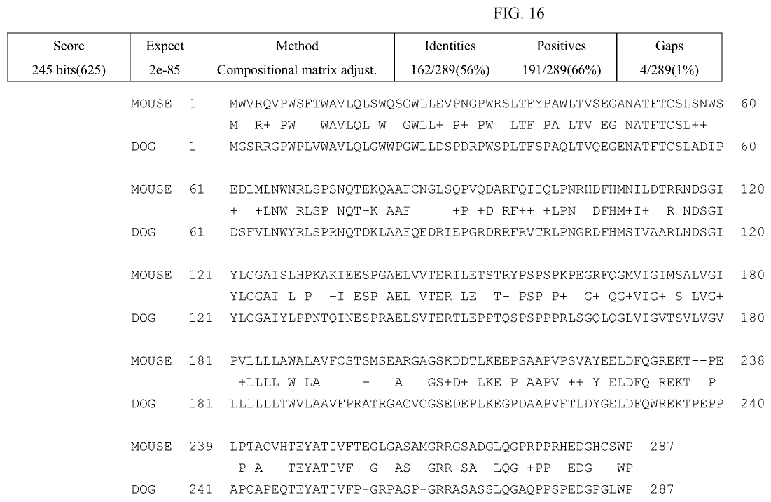

FIG. 16 shows the alignment between mouse PD-1 amino acid sequence (NP_032824.1; SEQ ID NO: 2) and canine PD-1 amino acid sequence (NP_001301026.1; SEQ ID NO: 4). Thus, the corresponding amino acid residue or region between canine and mouse PD-1 can be found in FIG. 16.

PD-1 genes, proteins, and locus of the other species are also known in the art. For example, the gene ID for PD-1 in Rattus norvegicus is 301626, the gene ID for PD-1 in Macaca mulatta (Rhesus monkey) is 100135775, and the gene ID for PD-1 in Bos taurus (cattle) is 613842. The relevant information for these genes (e.g., intron sequences, exon sequences, amino acid residues of these proteins) can be found, e.g., in NCBI database, which is incorporated by reference herein in its entirety.

The present disclosure provides canine or chimeric (e.g., caninized) PD-1 nucleotide sequence and/or amino acid sequences. In some embodiments, the entire sequence of mouse exon 1, exon 2, exon 3, exon 4, exon 5, signal peptide, extracellular region, transmembrane region, and/or cytoplasmic region are replaced by the corresponding canine sequence. In some embodiments, a “region” or “portion” of mouse exon 1, exon 2, exon 3, exon 4, exon 5, signal peptide, extracellular region, transmembrane region, and/or cytoplasmic region are replaced by the corresponding canine sequence. The term “region” or “portion” can refer to at least 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 500, or 600 nucleotides, or at least 1, 2, 3, 4, 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the “region” or “portion” can be at least 50%, 55%, 60%, 65%, 70%, 75%, 80%, 85%, 90%, 95%, or 99% identical to exon 1, exon 2, exon 3, exon 4, exon 5, signal peptide, extracellular region, transmembrane region, or cytoplasmic region. In some embodiments, a region, a portion, or the entire sequence of mouse exon 1, exon 2, exon 3, exon 4, and/or exon 5 (e.g., exon 2) are replaced by the canine exon 1, exon 2, exon 3, exon 4, and/or exon 5 (e.g., exon 2) sequence.

In some embodiments, the present disclosure also provides a chimeric (e.g., caninized) PD-1 nucleotide sequence and/or amino acid sequences, wherein in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from mouse PD-1 mRNA sequence (e.g., SEQ ID NO: 1), mouse PD-1 amino acid sequence (e.g., SEQ ID NO: 2), or a portion thereof (e.g., exon 1, exon 2, exon 3, exon 4, and exon 5); and in some embodiments, at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% of the sequence are identical to or derived from canine PD-1 mRNA sequence (e.g., SEQ ID NO: 3), canine PD-1 amino acid sequence (e.g., SEQ ID NO: 4), or a portion thereof (e.g., exon 1, exon 2, exon 3, exon 4, and exon 5).

In some embodiments, the chimeric PD-1 sequence encodes amino acids 1-30 and 142-288 of mouse PD-1 (SEQ ID NO: 2). In some embodiments, the chimeric PD-1 sequence encodes amino acids 31-141 of canine PD-1 (SEQ ID NO: 4).

In some embodiments, the nucleic acids as described herein are operably linked to a promotor or regulatory element, e.g., an endogenous mouse PD-1 promotor, an inducible promoter, an enhancer, and/or mouse or canine regulatory elements.

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that are different from a portion of or the entire mouse PD-1 nucleotide sequence (e.g., exon 1, exon 2, exon 3, exon 4, exon 5, or NM_008798.2 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire mouse PD-1 nucleotide sequence (e.g., exon 1, exon 2, exon 3, exon 4, exon 5, or NM_008798.2 (SEQ ID NO: 1)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from a portion of or the entire canine PD-1 nucleotide sequence (e.g., exon 1, exon 2, exon 3, exon 4, exon 5, or NM_001314097.1 (SEQ ID NO: 3)).

In some embodiments, the nucleic acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as a portion of or the entire canine PD-1 nucleotide sequence (e.g., exon 1, exon 2, exon 3, exon 4, exon 5, or NM_001314097.1 (SEQ ID NO: 3)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire mouse PD-1 amino acid sequence (e.g., exon 1, exon 2, exon 3, exon 4, exon 5, or NP_032824.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire mouse PD-1 amino acid sequence (e.g., exon 1, exon 2, exon 3, exon 4, exon 5, or NP_032824.1 (SEQ ID NO: 2)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from a portion of or the entire canine PD-1 amino acid sequence (e.g., exon 1, exon 2, exon 3, exon 4, exon 5, or NP_001301026.1 (SEQ ID NO: 4)).

In some embodiments, the amino acid sequence has at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as a portion of or the entire canine PD-1 amino acid sequence (e.g., exon 1, exon 2, exon 3, exon 4, exon 5, or NP_001301026.1 (SEQ ID NO: 4)).

The present disclosure also provides a caninized PD-1 mouse amino acid sequence, wherein the amino acid sequence is selected from the group consisting of:

a) an amino acid sequence shown in SEQ ID NO: 8;

b) an amino acid sequence having a homology of at least 90% with or at least 90% identical to the amino acid sequence shown in SEQ ID NO: 8 or SEQ ID NO: 4;

c) an amino acid sequence encoded by a nucleic acid sequence, wherein the nucleic acid sequence is able to hybridize to a nucleotide sequence encoding the amino acid shown in SEQ ID NO: 8 or SEQ ID NO: 4 under a low stringency condition or a strict stringency condition;

d) an amino acid sequence having a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the amino acid sequence shown in SEQ ID NO: 8 or SEQ ID NO: 4;

e) an amino acid sequence that is different from the amino acid sequence shown in SEQ ID NO: 8 or SEQ ID NO: 4 by no more than 10, 9, 8, 7, 6, 5, 4, 3, 2 or no more than 1 amino acid; or

f) an amino acid sequence that comprises a substitution, a deletion and/or insertion of one or more amino acids to the amino acid sequence shown in SEQ ID NO: 8 or SEQ ID NO: 4.

The present disclosure also relates to a PD-1 nucleic acid (e.g., DNA or RNA) sequence, wherein the nucleic acid sequence can be selected from the group consisting of:

a) a nucleic acid sequence as shown in SEQ ID NO: 5, SEQ ID NO: 6, or SEQ ID NO: 7, or a nucleic acid sequence encoding a homologous PD-1 amino acid sequence of a caninized mouse;

b) a nucleic acid sequence that is shown in SEQ ID NO: 5, SEQ ID NO: 6, or SEQ ID NO: 7;

c) a nucleic acid sequence that is able to hybridize to the nucleotide sequence as shown in SEQ ID NO: 5, SEQ ID NO: 6, or SEQ ID NO: 7 under a low stringency condition or a strict stringency condition;

d) a nucleic acid sequence that has a homology of at least 90%, 91%, 92%, 93%, 94%, 95%, 96%, 97%, 98%, or 99%, or at least 90%, 91%, 92%, 93%, 94%, 95%, 96%, 97%, 98%, or 99% identical to the nucleotide sequence as shown in SEQ ID NO: 5, SEQ ID NO: 6, or SEQ ID NO: 7;

e) a nucleic acid sequence that encodes an amino acid sequence, wherein the amino acid sequence has a homology of at least 90% with or at least 90% identical to the amino acid sequence shown in SEQ ID NO: 8 or SEQ ID NO: 4;

f) a nucleic acid sequence that encodes an amino acid sequence, wherein the amino acid sequence has a homology of at least 90%, 91%, 92%, 93%, 94%, 95%, 96%, 97%, 98%, or 99% with, or at least 90%, 91%, 92%, 93%, 94%, 95%, 96%, 97%, 98%, or 99% identical to the amino acid sequence shown in SEQ ID NO: 8 or SEQ ID NO: 4;

g) a nucleic acid sequence that encodes an amino acid sequence, wherein the amino acid sequence is different from the amino acid sequence shown in SEQ ID NO: 8 or SEQ ID NO: 4 by no more than 10, 9, 8, 7, 6, 5, 4, 3, 2 or no more than 1 amino acid; and/or

h) a nucleic acid sequence that encodes an amino acid sequence, wherein the amino acid sequence comprises a substitution, a deletion and/or insertion of one or more amino acids to the amino acid sequence shown in SEQ ID NO: 8 or SEQ ID NO: 4.

The present disclosure further relates to a PD-1 genomic DNA sequence of a caninized mouse. The DNA sequence is obtained by a reverse transcription of the mRNA obtained by transcription thereof is consistent with or complementary to the DNA sequence homologous to the sequence shown in SEQ ID NO: 5, SEQ ID NO: 6, or SEQ ID NO: 7.

The disclosure also provides an amino acid sequence that has a homology of at least 90% with, or at least 90% identical to the sequence shown in SEQ ID NO: 8 or SEQ ID NO: 4, and has protein activity. In some embodiments, the homology with the sequence shown in SEQ ID NO: 8 or SEQ ID NO: 4 is at least about 90%, 91%, 92%, 93%, 94%, 95%, 96%, 97%, 98%, or at least 99%. In some embodiments, the foregoing homology is at least about 60%, 61%, 62%, 63%, 64%, 65%, 66%, 67%, 68%, 69%, 70%, 71%, 72%, 73%, 74%, 75%, 80%, or 85%.

In some embodiments, the percentage identity with the sequence shown in SEQ ID NO: 8 or SEQ ID NO: 4 is at least about 90%, 91%, 92%, 93%, 94%, 95%, 96%, 97%, 98%, or at least 99%. In some embodiments, the foregoing percentage identity is at least about 60%, 61%, 62%, 63%, 64%, 65%, 66%, 67%, 68%, 69%, 70%, 71%, 72%, 73%, 74%, 75%, 80%, or 85%.

The disclosure also provides a nucleotide sequence that has a homology of at least 90%, or at least 90% identical to the sequence shown in SEQ ID NO: 5, SEQ ID NO:6, or SEQ ID NO: 7, and encodes a polypeptide that has protein activity. In some embodiments, the homology with the sequence shown in SEQ ID NO: 5, SEQ ID NO: 6, or SEQ ID NO: 7 is at least about 90%, 91%, 92%, 93%, 94%, 95%, 96%, 97%, 98%, or at least 99%. In some embodiments, the foregoing homology is at least about 50%, 55%, 60%, 65%, 66%, 67%, 68%, 69%, 70%, 71%, 72%, 73%, 74%, 75%, 80%, or 85%.

In some embodiments, the percentage identity with the sequence shown in SEQ ID NO: 5, SEQ ID NO: 6, or SEQ ID NO: 7 is at least about 90%, 91%, 92%, 93%, 94%, 95%, 96%, 97%, 98%, or at least 99%. In some embodiments, the foregoing percentage identity is at least about 50%, 55%, 60%, 65%, 66%, 67%, 68%, 69%, 70%, 71%, 72%, 73%, 74%, 75%, 80%, or 85%.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

The percentage of residues conserved with similar physicochemical properties (percent homology), e.g. leucine and isoleucine, can also be used to measure sequence similarity. Families of amino acid residues having similar physicochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

Cells, tissues, and animals (e.g., mouse) are also provided that comprise the nucleotide sequences as described herein, as well as cells, tissues, and animals (e.g., mouse) that express canine or chimeric (e.g., caninized) PD-1 from an endogenous non-canine PD-1 locus.

### Genetically Modified Animals

As used herein, the term “genetically-modified non-human animal” refers to a non-human animal having exogenous DNA in at least one chromosome of the animal's genome. The term “genetically-modified non-canine animal” refers to a non-canine animal having exogenous DNA in at least one chromosome of the animal's genome.

In some embodiments, at least one or more cells, e.g., at least 1%, 2%, 3%, 4%, 5%, 10%, 20%, 30%, 40%, 50% of cells of the genetically-modified animal have the exogenous DNA in its genome. The cell having exogenous DNA can be various kinds of cells, e.g., an endogenous cell, a somatic cell, an immune cell, a T cell, a B cell, an antigen presenting cell, a macrophage, a dendritic cell, a germ cell, a blastocyst, or an endogenous tumor cell. In some embodiments, genetically-modified animals are provided that comprise a modified endogenous PD-1 locus that comprises an exogenous sequence (e.g., a canine sequence), e.g., a replacement of one or more endogenous sequences with one or more canine sequences. The animals are generally able to pass the modification to progeny, i.e., through germline transmission.

As used herein, the term “chimeric gene” or “chimeric nucleic acid” refers to a gene or a nucleic acid, wherein two or more portions of the gene or the nucleic acid are from different species, or at least one of the sequences of the gene or the nucleic acid does not correspond to the wild-type nucleic acid in the animal. In some embodiments, the chimeric gene or chimeric nucleic acid has at least one portion of the sequence that is derived from two or more different sources, e.g., sequences encoding different proteins or sequences encoding the same (or homologous) protein of two or more different species. In some embodiments, the chimeric gene or the chimeric nucleic acid is a caninized gene or caninized nucleic acid.

As used herein, the term “chimeric protein” or “chimeric polypeptide” refers to a protein or a polypeptide, wherein two or more portions of the protein or the polypeptide are from different species, or at least one of the sequences of the protein or the polypeptide does not correspond to wild-type amino acid sequence in the animal. In some embodiments, the chimeric protein or the chimeric polypeptide has at least one portion of the sequence that is derived from two or more different sources, e.g., same (or homologous) proteins of different species. In some embodiments, the chimeric protein or the chimeric polypeptide is a caninized protein or a caninized polypeptide.

In some embodiments, the chimeric gene or the chimeric nucleic acid is a caninized PD-1 gene or a caninized PD-1 nucleic acid. In some embodiments, at least one or more portions of the gene or the nucleic acid is from the canine PD-1 gene, at least one or more portions of the gene or the nucleic acid is from an endogenous (e.g., mouse) PD-1 gene. In some embodiments, the gene or the nucleic acid comprises a sequence that encodes a PD-1 protein. The encoded PD-1 protein is functional or has at least one activity of the canine PD-1 protein or the endogenous (e.g., mouse) PD-1 protein, e.g., binding with canine or endogenous PD-L1 or PD-L2, decreasing the level of activation of immune cells (e.g., T cells), reducing apoptosis in regulatory T cells, promoting apoptosis in antigen-specific T-cells in lymph nodes, and/or downregulating the immune response.

In some embodiments, the chimeric protein or the chimeric polypeptide is a caninized PD-1 protein or a caninized PD-1 polypeptide. In some embodiments, at least one or more portions of the amino acid sequence of the protein or the polypeptide is from a canine PD-1 protein, and at least one or more portions of the amino acid sequence of the protein or the polypeptide is from an endogenous PD-1 protein. The caninized PD-1 protein or the caninized PD-1 polypeptide is functional or has at least one activity of the canine PD-1 protein or the endogenous PD-1 protein.

The genetically modified animal can be various animals, e.g., a mouse, rat, rabbit, pig, bovine (e.g., cow, bull, buffalo), deer, sheep, goat, chicken, cat, ferret, primate (e.g., marmoset, rhesus monkey). For the animals where suitable genetically modifiable embryonic stem (ES) cells are not readily available, other methods are employed to make an animal comprising the genetic modification. Such methods include, e.g., modifying a non-ES cell genome (e.g., a fibroblast or an induced pluripotent cell) and employing nuclear transfer to transfer the modified genome to a suitable cell, e.g., an oocyte, and gestating the modified cell (e.g., the modified oocyte) in an animal under suitable conditions to form an embryo. These methods are known in the art, and are described, e.g., in A. Nagy, et al., “Manipulating the Mouse Embryo: A Laboratory Manual (Third Edition),” Cold Spring Harbor Laboratory Press, 2003, which is incorporated by reference herein in its entirety.

In one aspect, the animal is a mammal, e.g., of the superfamily Dipodoidea or Muroidea. In some embodiments, the genetically modified animal is a rodent. The rodent can be selected from a mouse, a rat, and a hamster. In some embodiments, the genetically modified animal is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, with-tailed rats, malagasy rats and mice), Platacanthomyidae (e.g., spiny dormice), and Spalacidae (e.g., mole rates, bamboo rats, and zokors). In some embodiments, the genetically modified rodent is selected from a true mouse or rat (family Muridae), a gerbil, a spiny mouse, and a crested rat. In some embodiments, the animal is a mouse.

In some embodiments, the animal is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the mouse is a 129 strain selected from the group consisting of a strain that is 129P1, 129P2, 129P3, 129X1, 129S1 (e.g., 129S1/SV, 129S1/SvIm), 129S2, 129S4, 129S5, 129S9/SvEvH, 129S6 (129/SvEvTac), 129S7, 129S8, 129T1, 129T2. These mice are described, e.g., in Festing et al., Revised nomenclature for strain 129 mice, Mammalian Genome 10: 836 (1999); Auerbach et al., Establishment and Chimera Analysis of 129/SvEv- and C57BL/6-Derived Mouse Embryonic Stem Cell Lines (2000), both of which are incorporated herein by reference in the entirety. In some embodiments, the genetically modified mouse is a mix of the 129 strain and the C57BL/6 strain. In some embodiments, the mouse is a mix of the 129 strains, or a mix of the BL/6 strains. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129).

In some embodiments, the animal is a rat. The rat can be selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

The animal can have one or more other genetic modifications, and/or other modifications, that are suitable for the particular purpose for which the caninized PD-1 animal is made. For example, suitable mice for maintaining a xenograft (e.g., a canine cancer or tumor), can have one or more modifications that compromise, inactivate, or destroy the immune system of the animal in whole or in part. Compromise, inactivation, or destruction of the immune system of the animal can include, for example, destruction of hematopoietic cells and/or immune cells by chemical means (e.g., administering a toxin), physical means (e.g., irradiating the animal), and/or genetic modification (e.g., knocking out one or more genes). Non-limiting examples of such mice include, e.g., NOD mice, SCID mice, NOD/SCID mice, IL2Ry knockout mice, NOD/SCID/γc null mice, nude mice, and Rag1 and/or Rag2 knockout mice. These mice can optionally be irradiated, or otherwise treated to destroy one or more immune cell type. Thus, in various embodiments, a genetically modified mouse is provided that can include a caninization of at least a portion of an endogenous PD-1 locus, and further comprises a modification that compromises, inactivates, or destroys the immune system (or one or more cell types of the immune system) of the animal in whole or in part. In some embodiments, modification is, e.g., selected from the group consisting of a modification that results in NOD mice, SCID mice, NOD/SCID mice, IL-2Ry knockout mice, NOD/SCID/γc null mice, nude mice, Rag1 and/or Rag2 knockout mice, and a combination thereof. These genetically modified animals are described, e.g., in US20150106961, which is incorporated herein by reference in its entirety. In some embodiments, the mouse can include a replacement of all or part of mature PD-1 coding sequence with canine mature PD-1 coding sequence or an insertion of canine mature PD-1 coding sequence or chimeric PD-1 coding sequence.

Genetically modified animals that comprise a modification of an endogenous PD-1 locus. In some embodiments, the modification can comprise a nucleic acid sequence encoding at least a portion of a mature PD-1 protein (e.g., at least 10%, 20%, 30%, 40%, 50%, 60%, 70%, 80%, 90%, 95%, 96%, 97%, 98%, or 99% identical to the mature PD-1 protein sequence). Although genetically modified cells are also provided that can comprise the modifications described herein (e.g., ES cells, somatic cells), in many embodiments, the genetically modified animals comprise the modification of the endogenous PD-1 locus in the germline of the animal.

Genetically modified animals can express a canine PD-1 and/or a chimeric (e.g., caninized) PD-1 from endogenous mouse loci, wherein the endogenous mouse PD-1 gene has been replaced with a canine PD-1 gene and/or a nucleotide sequence that encodes a region of canine PD-1 sequence or an amino acid sequence that is at least 10%, 20%, 30%, 40%, 50%, 60%, 70&, 80%, 90%, 95%, 96%, 97%, 98%, or 99% identical to the canine PD-1 sequence. In various embodiments, an endogenous PD-1 locus is modified in whole or in part to comprise canine nucleic acid sequence encoding at least one protein-coding sequence of a mature PD-1 protein.

In some embodiments, the genetically modified mice express the canine PD-1 and/or chimeric PD-1 (e.g., caninized PD-1) from endogenous loci that are under control of mouse promoters and/or mouse regulatory elements. The replacement(s) at the endogenous mouse loci provide animals that express canine PD-1 or chimeric PD-1 (e.g., caninized PD-1) in appropriate cell types and in a manner that does not result in the potential pathologies observed in some other transgenic mice known in the art. The canine PD-1 or the chimeric PD-1 (e.g., caninized PD-1) expressed in animal can maintain one or more functions of the wild-type mouse or canine PD-1 in the animal. For example, canine or murine PD-1 ligands (e.g., PD-L1 or PD-L2) can bind to the expressed PD-1, downregulate immune response, e.g., downregulate immune response by at least 10%, 20%, 30%, 40%, or 50%. Furthermore, in some embodiments, the animal does not express endogenous PD-1. As used herein, the term “endogenous PD-1” refers to PD-1 protein that is expressed from an endogenous PD-1 nucleotide sequence of the animal (e.g., mouse) before any genetic modification.

The genome of the animal can comprise a sequence encoding an amino acid sequence that is at least 70%, 75%, 80%, 85%, 90%, 95%, 99%, or 100% identical to canine PD-1 (NP_001301026.1) (SEQ ID NO: 4). In some embodiments, the genome comprises a sequence encoding an amino acid sequence that is at least 70%, 75%, 80%, 85%, 90%, 95%, 99%, or 100% identical to SEQ ID NO: 8.

The genome of the genetically modified animal can comprise a replacement at an endogenous PD-1 gene locus of a sequence encoding a region of endogenous PD-1 with a sequence encoding a corresponding region of canine PD-1. In some embodiments, the sequence that is replaced is any sequence within the endogenous PD-1 gene locus, e.g., exon 1, exon 2, exon 3, exon 4, exon 5, 5′-UTR, 3′-UTR, the first intron, the second intron, the third intron, and the fourth intron, etc. In some embodiments, the sequence that is replaced is within the regulatory region of the endogenous PD-1 gene. In some embodiments, the sequence that is replaced is exon 2 or part thereof, of an endogenous mouse PD-1 gene locus.

In some embodiments, a sequence that encodes an amino acid sequence (e.g., canine PD-1 or chimeric PD-1) is inserted after the start codon (e.g., within 10, 20, 30, 40, 50, 60, 70, 80, 90, or 100 nucleic acids). The start codon is the first codon of a messenger RNA (mRNA) transcript translated by a ribosome. The start codon always codes for methionine in eukaryotes and a modified Met (fMet) in prokaryotes. The most common start codon is ATG (or AUG in mRNA).

In some embodiments, the inserted sequence further comprises a stop codon (e.g., TAG, TAA, TGA). The stop codon (or termination codon) is a nucleotide triplet within messenger RNA that signals a termination of translation into proteins. Thus, the endogenous sequence after the stop codon will not be translated into proteins. In some embodiments, at least one exons of (e.g., exon 1, exon 2, exon 3, exon 4, and/or exon 5) of the endogenous PD-1 gene are not translated into proteins.

The genetically modified animal can have one or more cells expressing a canine or chimeric PD-1 (e.g., caninized PD-1) having an extracellular region and a cytoplasmic region, wherein the extracellular region comprises a sequence that is at least 50%, 60%, 70%, 80%, 90%, 95%, 99% identical to the extracellular region of canine PD-1. In some embodiments, the extracellular region of the caninized PD-1 has a sequence that has at least 10, 20, 30, 40, 50, 60, 70, 80, 90, 100, 110, 120, 130, 140, 150, 160, 170, or 180 amino acids (e.g., contiguously or non-contiguously) that are identical to canine PD-1. Because canine PD-1 and endogenous PD-1 (e.g., mouse PD-1) sequences, in many cases, are different, antibodies that bind to canine PD-1 will not necessarily have the same binding affinity with endogenous PD-1 or have the same effects to endogenous PD-1. Therefore, the genetically modified animal having a canine or a caninized extracellular region can be used to better evaluate the effects of anti-canine PD-1 antibodies in an animal model. In some embodiments, the genome of the genetically modified animal comprises a sequence encoding an amino acid sequence that corresponds to part or the entire sequence of exon 1, exon 2, exon 3, exon 4, and/or exon 5 of canine PD-1, part or the entire sequence of extracellular region of canine PD-1 (with or without signal peptide), or part or the entire sequence of amino acids 31-141 of SEQ ID NO: 4.

In some embodiments, the animal can have, at an endogenous PD-1 gene locus, a nucleotide sequence encoding a chimeric canine/mouse PD-1 polypeptide, wherein a canine portion of the chimeric canine/mouse PD-1 polypeptide comprises a portion of canine PD-1 extracellular domain, and wherein the animal expresses a functional PD-1 on a surface of a cell of the animal. The canine portion of the chimeric canine/mouse PD-1 polypeptide can comprise a portion of exon 1, exon 2, exon 3, exon 4, and/or exon 5 of canine PD-1. In some embodiments, the canine portion of the chimeric canine/mouse PD-1 polypeptide can comprise a sequence that is at least 80%, 85%, 90%, 95%, or 99% identical to amino acids 31-141 of SEQ ID NO: 4.

In some embodiments, the mouse portion of the chimeric canine/mouse PD-1 polypeptide comprises transmembrane and/or cytoplasmic regions of an endogenous mouse PD-1 polypeptide. There may be several advantages that are associated with the transmembrane and/or cytoplasmic regions of an endogenous mouse PD-1 polypeptide. For example, once a PD-1 ligand (e.g., PD-L1) or an anti-PD-1 antibody binds to PD-1, they can properly transmit extracellular signals into the cells and initiate the downstream pathway. A canine or caninized transmembrane and/or cytoplasmic regions may not function properly in non-canine animal cells. In some embodiments, a few extracellular amino acids that are close to the transmembrane region of PD-1 are also derived from endogenous sequence. These amino acids can also be important for transmembrane signal transmission.

Furthermore, the genetically modified animal can be heterozygous with respect to the replacement or insertion at the endogenous PD-1 locus, or homozygous with respect to the replacement or insertion at the endogenous PD-1 locus.

In some embodiments, the genetically modified animal (e.g., a rodent) comprises a caninization of an endogenous PD-1 gene, wherein the caninization comprises a replacement at the endogenous rodent PD-1 locus of a nucleic acid comprising an exon of a PD-1 gene with a nucleic acid sequence comprising at least one exon of a canine PD-1 gene to form a modified PD-1 gene.

In some embodiments, the genetically modified animal (e.g., a rodent) comprises an insertion at the endogenous rodent PD-1 locus of a nucleic acid sequence comprising at least one exon of a canine PD-1 gene to form a modified PD-1 gene.

In some embodiments, the expression of the modified PD-1 gene is under control of regulatory elements at the endogenous PD-1 locus.

In some embodiments, the caninized PD-1 locus lacks a canine PD-1 5′-UTR. In some embodiment, the caninized PD-1 locus comprises a rodent (e.g., mouse) 5′-UTR. In some embodiments, the caninization comprises a canine 3′-UTR. In appropriate cases, it may be reasonable to presume that the mouse and canine PD-1 genes appear to be similarly regulated based on the similarity of their 5′-flanking sequence. As shown in the present disclosure, caninized PD-1 mice that comprise a replacement at an endogenous mouse PD-1 locus, which retain mouse regulatory elements but comprise a caninization of PD-1 encoding sequence, do not exhibit obvious pathologies. Both genetically modified mice that are heterozygous or homozygous for caninized PD-1 are grossly normal.

The present disclosure further relates to a mammal generated through any methods described herein. In some embodiments, the genome thereof contains canine genes or caninized genes.

In some embodiments, the mammal is a rodent, and preferably, the mammal is a mouse.

In some embodiments, the mammal expresses a protein encoded by a caninized PD-1 gene.

In addition, the present disclosure also relates to a tumor bearing mammal model, characterized in that the mammal model is obtained through the methods as described herein. In some embodiments, the mammal is a rodent (e.g., a mouse).

The present disclosure further relates to a cell or cell line, or a primary cell culture thereof derived from the mammal or an offspring thereof, or the tumor bearing mammal; the tissue, organ or a culture thereof derived from the mammal or an offspring thereof, or the tumor bearing mammal; and the tumor tissue derived from the mammal or an offspring thereof when it bears a tumor, or the tumor bearing mammal.

The present disclosure also provides mammals produced by any of the methods described herein. In some embodiments, a mammal is provided; and the genetically modified animal contains the DNA encoding canine or caninized PD-1 in the genome of the animal.

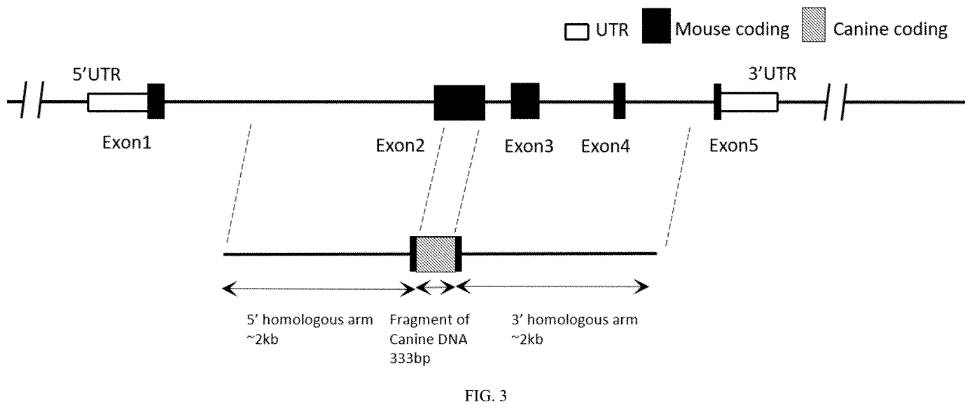

In some embodiments, the mammal comprises the genetic construct as described herein (e.g., gene construct as shown in FIG. 2 or FIG. 3). In some embodiments, a mammal expressing canine or caninized PD-1 is provided. In some embodiments, the tissue-specific expression of canine or caninized PD-1 protein is provided.

In some embodiments, the expression of canine or caninized PD-1 in a genetically modified animal is controllable, as by the addition of a specific inducer or repressor sub stance.

Genetic, molecular and behavioral analyses for the mammals described above can be performed. The present disclosure also relates to the progeny produced by the mammal provided by the present disclosure mated with the same or other genotypes.

The present disclosure also provides a cell line or primary cell culture derived from the mammal or a progeny thereof. A model based on cell culture can be prepared, for example, by the following methods. Cell cultures can be obtained by way of isolation from a mammal, alternatively cell can be obtained from the cell culture established using the same constructs and the standard cell transfection techniques. The integration of genetic constructs containing DNA sequences encoding canine PD-1 protein or chimeric PD-1 protein can be detected by a variety of methods.

There are many analytical methods that can be used to detect exogenous DNA, including methods at the level of nucleic acid (including the mRNA quantification approaches using reverse transcriptase polymerase chain reaction (RT-PCR) or Southern blotting, and in situ hybridization) and methods at the protein level (including histochemistry, immunoblot analysis and in vitro binding studies). In addition, the expression level of the gene of interest can be quantified by ELISA techniques well known to those skilled in the art. Many standard analysis methods can be used to complete quantitative measurements. For example, transcription levels can be measured using RT-PCR and hybridization methods including RNase protection, Southern blot analysis, RNA dot analysis (RNAdot) analysis. Immunohistochemical staining, flow cytometry, Western blot analysis can also be used to assess the presence of canine or caninized PD-1 protein.

### Vectors

The disclosure also provides vectors for constructing a caninized PD-1 animal model or a knock-out model. In some embodiments, the vectors comprise sgRNA sequence, wherein the sgRNA sequence target PD-1 gene, and the sgRNA is unique on the target sequence of the PD-1 gene to be altered, and meets the sequence arrangement rule of 5′-NNN (20)-NGG3′ or 5′-CCN-N(20)-3′; and in some embodiments, the targeting site of the sgRNA in the mouse PD-1 gene is located on the exon 1, exon 2, exon 3, exon 4, exon 5, intron 1, intron 2, intron 3, intron 4, upstream of exon 1, or downstream of exon 5 of the mouse PD-1 gene.

In some embodiments, the 5′ targeting sequence for the sequence is shown as SEQ ID NOS: 18-21, and the sgRNA sequence recognizes the 5′ targeting site. In some embodiments, the 3′ targeting sequence for the knockout sequence is shown as SEQ ID NOS: 22-25 and the sgRNA sequence recognizes the 3′ targeting site. Thus, the disclosure provides sgRNA sequences for constructing a genetic modified animal model. In some embodiments, the oligonucleotide sgRNA sequences are set forth in SEQ ID NOS: 26-33.

In some embodiments, the disclosure relates to a plasmid construct (e.g., pT7-sgRNA) including the sgRNA sequence, and/or a cell including the construct.

The present disclosure also provides a targeting vector, comprising: a) a DNA fragment homologous to the 5′ end of a region to be altered (5′ arm), which is selected from the PD-1 gene genomic DNAs in the length of 100 to 10,000 nucleotides; b) a desired/donor DNA sequence encoding a donor region; and c) a second DNA fragment homologous to the 3′ end of the region to be altered (3′ arm), which is selected from the PD-1 gene genomic DNAs in the length of 100 to 10,000 nucleotides.

In some embodiments, a) the DNA fragment homologous to the 5′ end of a conversion region to be altered (5′ arm) is selected from the nucleotide sequences that have at least 90% homology to the NCBI accession number NC_000067.6; c) the DNA fragment homologous to the 3′ end of the region to be altered (3′ arm) is selected from the nucleotide sequences that have at least 90% homology to the NCBI accession number NC_000067.6.

In some embodiments, a) the DNA fragment homologous to the 5′ end of a region to be altered (5′ arm) is selected from the nucleotides from the position 94041502 to the position 94043271 of the NCBI accession number NC_000067.6; c) the DNA fragment homologous to the 3′ end of the region to be altered (3′ arm) is selected from the nucleotides from the position 94039436 to the position 94041168 of the NCBI accession number NC_000067.6.

In some embodiments, the length of the selected genomic nucleotide sequence in the targeting vector can be more than about 300 bp, 400 bp, 500 bp, or 1 kb.

In some embodiments, the region to be altered is exon 1, exon 2, exon 3, exon 4, and/or exon 5 of PD-1 gene (e.g., exon 2 of mouse PD-1 gene).

The targeting vector can further include a selected gene marker.

In some embodiments, the sequence of the 5′ arm is shown in SEQ ID NO: 9; and the sequence of the 3′ arm is shown in SEQ ID NO: 10.

In some embodiments, the sequence is derived from a canine sequence. For example, the target region in the targeting vector is a part or entirety of the nucleotide sequence of a canine PD-1 or a chimeric PD-1. In some embodiments, the nucleotide sequence of the caninized PD-1 encodes the entire or the part of canine PD-1 protein with the NCBI accession number NP_001301026.1 (SEQ ID NO: 4).

The disclosure also relates to a cell comprising the vectors as described above.

In addition, the present disclosure further relates to a mammalian cell, having any one of the foregoing targeting vectors, and one or more in vitro transcripts of the construct as described herein. In some embodiments, the cell includes Cas9 mRNA or an in vitro transcript thereof.

In some embodiments, the genes in the cell are heterozygous. In some embodiments, the genes in the cell are homozygous.

In some embodiments, the mammalian cell is a mouse cell. In some embodiments, the cell is a fertilized egg cell.

### Methods of Making Genetically Modified Animals

Genetically modified animals can be made by several techniques that are known in the art, including, e.g., nonhomologous end-joining (NHEJ), homologous recombination (HR), zinc finger nucleases (ZFNs), transcription activator-like effector-based nucleases (TALEN), and the clustered regularly interspaced short palindromic repeats (CRISPR)-Cas system. In some embodiments, homologous recombination is used. In some embodiments, CRISPR-Cas9 genome editing is used to generate genetically modified animals. Many of these genome editing techniques are known in the art, and is described, e.g., in Yin et al., “Delivery technologies for genome editing,” Nature Reviews Drug Discovery 16.6 (2017): 387-399, which is incorporated by reference in its entirety. Many other methods are also provided and can be used in genome editing, e.g., micro-injecting a genetically modified nucleus into an enucleated oocyte, and fusing an enucleated oocyte with another genetically modified cell.

Thus, in some embodiments, the disclosure provides replacing in at least one cell of the animal, at an endogenous PD-1 gene locus, a sequence encoding a region of an endogenous PD-1 with a sequence encoding a corresponding region of canine PD-1, a sequencing encoding canine PD-1, or a sequencing encoding chimeric PD-1.

In some embodiments, the disclosure provides inserting in at least one cell of the animal, at an endogenous PD-1 gene locus, a sequence encoding a canine PD-1 or a chimeric PD-1.

In some embodiments, the genetic modification occurs in a germ cell, a somatic cell, a blastocyst, or a fibroblast, etc. The nucleus of a somatic cell or the fibroblast can be inserted into an enucleated oocyte.

FIG. 3 show a caninization strategy for a mouse PD-1 locus. In FIG. 3, the targeting strategy involves a vector comprising the 5′ end homologous arm, canine PD-1 gene fragment or chimeric PD-1 gene fragment, 3′ homologous arm. The process can involve replacing endogenous PD-1 sequence with canine sequence by homologous recombination. In some embodiments, the cleavage at the upstream and the downstream of the target site (e.g., by zinc finger nucleases, TALEN or CRISPR) can result in DNA double strands break, and the homologous recombination is used to replace endogenous PD-1 sequence with canine PD-1 sequence.

Thus, in some embodiments, the methods for making a genetically modified, caninized animal, can include the step of replacing at an endogenous PD-1 locus (or site), a nucleic acid encoding a sequence encoding a region of endogenous PD-1 with a sequence encoding a canine PD-1 or a chimeric PD-1. The sequence can include a region (e.g., a part or the entire region) of exon 1, exon 2, exon 3, exon 4, exon 5 of a canine PD-1 gene. In some embodiments, the sequence includes a region of exon 1, exon 2, exon 3, exon 4, exon 5 of a canine PD-1 gene (e.g., amino acids 31-141 of SEQ ID NO: 4). In some embodiments, the region is located within the extracellular region of PD-1. In some embodiments, the endogenous PD-1 locus is exon 1, exon 2, exon 3, exon 4, and/or exon 5 of mouse PD-1 (e.g., exon 2).

In some embodiments, the methods of modifying a PD-1 locus of a mouse to express a chimeric canine/mouse PD-1 peptide can include the steps of replacing at the endogenous mouse PD-1 locus a nucleotide sequence encoding a mouse PD-1 with a nucleotide sequence encoding a canine PD-1, thereby generating a sequence encoding a chimeric canine/mouse PD-1.

In some embodiments, the nucleotide sequence encoding the chimeric canine/mouse PD-1 can include a first nucleotide sequence encoding an extracellular region of mouse PD-1 (with or without the mouse or canine signal peptide sequence); a second nucleotide sequence encoding an extracellular region of canine PD-1; a third nucleotide sequence encoding a transmembrane and a cytoplasmic region of a mouse PD-1.

In some embodiments, the nucleotide sequences as described herein do not overlap with each other (e.g., the first nucleotide sequence, the second nucleotide sequence, and/or the third nucleotide sequence do not overlap). In some embodiments, the amino acid sequences as described herein do not overlap with each other.

The present disclosure further provides a method for establishing a PD-1 gene caninized animal model, involving the following steps:

(a) providing the cell (e.g. a fertilized egg cell) based on the methods described herein;

(b) culturing the cell in a liquid culture medium;

(c) transplanting the cultured cell to the fallopian tube or uterus of the recipient female mammal, allowing the cell to develop in the uterus of the female mammal;

(d) identifying the germline transmission in the offspring genetically modified caninized mammal of the pregnant female in step (c).

In some embodiments, the mammal in the foregoing method is a mouse (e.g., a C57BL/6 mouse).

In some embodiments, the mammal in step (c) is a female with pseudo pregnancy (or false pregnancy).

In some embodiments, the fertilized eggs for the methods described above are C57BL/6 fertilized eggs. Other fertilized eggs that can also be used in the methods as described herein include, but are not limited to, FVB/N fertilized eggs, BALB/c fertilized eggs, DBA/1 fertilized eggs and DBA/2 fertilized eggs.

Fertilized eggs can come from any animal, e.g., any animal as described herein. In some embodiments, the fertilized egg cells are derived from rodents. The genetic construct can be introduced into a fertilized egg by microinjection of DNA. For example, by way of culturing a fertilized egg after microinjection, a cultured fertilized egg can be transferred to a false pregnant animal, which then gives birth of a mammal, so as to generate the mammal mentioned in the methods described above.

### Methods of Using Genetically Modified Animals

Replacement of genes in a non-canine animal with homologous or orthologous canine genes or canine sequences, at the endogenous locus and under control of endogenous promoters and/or regulatory elements, can result in an animal with qualities and characteristics that may be substantially different from a typical knockout-plus-transgene animal. In the typical knockout-plus-transgene animal, an endogenous locus is removed or damaged and a fully canine transgene is inserted into the animal's genome and presumably integrates at random into the genome. Typically, the location of the integrated transgene is unknown; expression of the canine protein is measured by transcription of the canine gene and/or protein assay and/or functional assay. Inclusion in the canine transgene of upstream and/or downstream canine sequences are apparently presumed to be sufficient to provide suitable support for expression and/or regulation of the transgene.

In some cases, the transgene with canine regulatory elements expresses in a manner that is unphysiological or otherwise unsatisfactory, and can be actually detrimental to the animal. The disclosure demonstrates that a replacement with canine sequence at an endogenous locus under control of endogenous regulatory elements provides a physiologically appropriate expression pattern and level that results in a useful caninized animal whose physiology with respect to the replaced gene are meaningful and appropriate in the context of the animal's physiology.

Genetically modified animals that express canine or caninized PD-1 protein, e.g., in a physiologically appropriate manner, provide a variety of uses that include, but are not limited to, developing therapeutics for canine diseases and disorders, and assessing the toxicity and/or the efficacy of these canine therapeutics in the animal models.

In various aspects, genetically modified animals are provided that express canine or caninized PD-1, which are useful for testing agents that can decrease or block the interaction between PD-1 and PD-1 ligands (e.g., PD-L1 or PD-L2) or the interaction between PD-1 and anti-canine PD-1 antibodies, testing whether an agent can increase or decrease the immune response, and/or determining whether an agent is an PD-1 agonist or antagonist. The genetically modified animals can be, e.g., an animal model of a canine disease, e.g., the disease is induced genetically (a knock-in or knockout). In various embodiments, the genetically modified animals further comprise an impaired immune system, e.g., an animal genetically modified to sustain or maintain a xenograft, e.g., a canine solid tumor or a blood cell tumor (e.g., a lymphocyte tumor, e.g., a B or T cell tumor).

In some embodiments, the genetically modified animals can be used for determining effectiveness of a PD-1 inhibitor for the treatment of cancer. The methods involve administering the PD-1 inhibitor (e.g., anti-canine PD-1 antibody or anti-canine PD-L1 antibody) to the animal as described herein, wherein the animal has a tumor; and determining the inhibitory effects of the PD-1 inhibitor to the tumor. In some embodiments, the PD-1 inhibitor is an anti-canine PD-1 antibody or anti-canine PD-L1 antibody.

The inhibitory effects that can be determined include, e.g., a decrease of tumor size or tumor volume, a decrease of tumor growth, a reduction of the increase rate of tumor volume in a subject (e.g., as compared to the rate of increase in tumor volume in the same subject prior to treatment or in another subject without such treatment), a decrease in the risk of developing a metastasis or the risk of developing one or more additional metastasis, an increase of survival rate, and an increase of life expectancy, etc. The tumor volume in a subject can be determined by various methods, e.g., as determined by direct measurement, MM or CT.

In some embodiments, the tumor comprises one or more cancer cells (e.g., canine or mouse cancer cells) that are injected into the animal. In some embodiments, the anti-PD-1 antibody, anti-PD-L1 antibody or anti-PD-L2 antibody prevents PD-1 ligands from binding to PD-1. In some embodiments, the anti-PD-1 antibody, anti-PD-L1 antibody, or anti-PD-L2 antibody does not prevent the ligands from binding to PD-1.

In some embodiments, the genetically modified animals can be used for determining whether an anti-PD-1 antibody is a PD-1 agonist or antagonist. In some embodiments, the methods as described herein are also designed to determine the effects of the agent (e.g., anti-PD-1 antibodies) on PD-1, e.g., whether the agent can stimulate immune cells or inhibit immune cells (e.g., T cells), whether the agent can increase or decrease the production of cytokines, whether the agent can activate or deactivate immune cells (e.g., T cells, macrophages, B cells, or DC), whether the agent can upregulate the immune response or downregulate immune response, and/or whether the agent can induce complement mediated cytotoxicity (CMC) or antibody dependent cellular cytoxicity (ADCC). In some embodiments, the genetically modified animals can be used for determining the effective dosage of a therapeutic agent for treating a disease in the subject, e.g., cancer, or autoimmune diseases.

The inhibitory effects on tumors can also be determined by methods known in the art, e.g., measuring the tumor volume in the animal, and/or determining tumor (volume) inhibition rate (TGI_{TV}). The tumor growth inhibition rate can be calculated using the formula TGI_{TV} (%)=(1−TVt/TVc)×100, where TVt and TVc are the mean tumor volume (or weight) of treated and control groups.

In some embodiments, the anti-PD-1 antibody or the anti-PD-L1 antibody is designed for treating various cancers. As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, the anti-PD-1 antibody is designed for treating melanoma (e.g., advanced melanoma), non-small cell lung carcinoma (NSCLC), small cell lung cancer (SCLC), B-cell non-Hodgkin lymphoma, bladder cancer, and/or prostate cancer (e.g., metastatic hormone-refractory prostate cancer). In some embodiments, the anti-PD-1 antibody is designed for treating hepatocellular, ovarian, colon, or cervical carcinomas. In some embodiments, the anti-PD-1 antibody is designed for treating advanced breast cancer, advanced ovarian cancer, and/or advanced refractory solid tumor. In some embodiments, the anti-PD-1 antibody is designed for treating metastatic solid tumors, NSCLC, melanoma, non-Hodgkin lymphoma, colorectal cancer, and multiple myeloma. In some embodiments, the anti-PD-1 antibody is designed for treating melanoma, pancreatic carcinoma, mesothelioma, hematological malignancies (e.g., Non-Hodgkin's lymphoma, lymphoma, chronic lymphocytic leukemia), or solid tumors (e.g., advanced solid tumors). In some embodiments, the anti-PD-1 antibody is designed for treating carcinomas (e.g., nasopharynx carcinoma, bladder carcinoma, cervix carcinoma, kidney carcinoma or ovary carcinoma).

In some embodiments, the anti-PD-1 antibody is designed for treating various autoimmune diseases. Thus, the methods as described herein can be used to determine the effectiveness of an anti-PD-1 antibody in inhibiting immune response.

The present disclosure also provides methods of determining toxicity of an antibody (e.g., anti-PD-1 antibody). The methods involve administering the antibody to the animal as described herein. The animal is then evaluated for its weight change, red blood cell count, hematocrit, and/or hemoglobin. In some embodiments, the antibody can decrease the red blood cells (RBC), hematocrit, or hemoglobin by more than 20%, 30%, 40%, or 50%. In some embodiments, the animals can have a weight that is at least 5%, 10%, 20%, 30%, or 40% smaller than the weight of the control group (e.g., average weight of the animals that are not treated with the antibody).

The present disclosure also relates to the use of the animal model generated through the methods as described herein in the development of a product related to an immunization process of cells, the manufacturing of a canine antibody, or the model system for a research in pharmacology, immunology, microbiology and medicine.

In some embodiments, the disclosure provides the use of the animal model generated through the methods as described herein in the production and utilization of an animal experimental disease model of an immunization processes, the study on a pathogen, or the development of a new diagnostic strategy and/or a therapeutic strategy.

The disclosure also relates to the use of the animal model generated through the methods as described herein in the screening, verifying, evaluating or studying the PD-1 gene function, canine PD-1 antibodies, drugs for canine PD-1 targeting sites, the drugs or efficacies for canine PD-1 targeting sites, the drugs for immune-related diseases and antitumor drugs.

Genetically Modified Animal Model with Two or More Canine or Chimeric Genes

The present disclosure further relates to methods for generating genetically modified animal model with two or more canine or chimeric genes. The animal can comprise a canine or chimeric PD-1 gene and a sequence encoding an additional canine or chimeric protein.

In some embodiments, the additional canine or chimeric protein can be cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD3, CD27, CD28, CD40, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), SIRPA, or TNF Receptor Superfamily Member 4 (TNFRSF4 or OX40).

The methods of generating genetically modified animal model with two or more canine or chimeric genes (e.g., caninized genes) can include the following steps:

(a) using the methods of introducing canine PD-1 gene or chimeric PD-1 gene as described herein to obtain a genetically modified animal;

(b) mating the genetically modified animal with another genetically modified animal, and then screening the progeny to obtain a genetically modified animal with two or more canine or chimeric genes.

In some embodiments, in step (b) of the method, the genetically modified animal can be mated with a genetically modified animal with canine or chimeric CTLA-4, LAG-3, BTLA, PD-L1, CD27, CD28, CD40, CD47, CD137, CD154, TIGIT, TIM-3, GITR, SIRPa, or OX40.

In some embodiments, the PD-1 caninization is directly performed on a genetically modified animal having a canine or chimeric CTLA-4, BTLA, PD-L1, CD3, CD27, CD28, CD40, CD47, CD137, CD154, TIGIT, TIM-3, GITR, SIRPa, or OX40 gene.

As these proteins may involve different mechanisms, a combination therapy that targets two or more of these proteins thereof may be a more effective treatment. In fact, many related clinical trials are in progress and have shown a good effect. The genetically modified animal model with two or more canine or caninized genes can be used for determining effectiveness of a combination therapy that targets two or more of these proteins, e.g., an anti-PD-1 antibody and an additional therapeutic agent for the treatment of cancer. The methods include administering the anti-PD-1 antibody and the additional therapeutic agent to the animal, wherein the animal has a tumor; and determining the inhibitory effects of the combined treatment to the tumor. In some embodiments, the additional therapeutic agent is an antibody that specifically binds to CTLA-4, BTLA, PD-L1, CD3, CD27, CD28, CD40, CD47, CD137, CD154, TIGIT, TIM-3, GITR, SIRPa, or OX40. In some embodiments, the additional therapeutic agent is an anti-CTLA4 antibody, an anti-PD-1 antibody, or an anti-PD-L1 antibody.

In some embodiments, the animal further comprises a sequence encoding a canine or caninized PD-L1, or a sequence encoding a canine or caninized CTLA-4. In some embodiments, the additional therapeutic agent is an anti-PD-L1 antibody, or an anti-CTLA-4 antibody. In some embodiments, the tumor comprises one or more tumor cells that express CD80, CD86, PD-L1, and/or PD-L2.

In some embodiments, the combination treatment is designed for treating various cancer as described herein, e.g., melanoma, non-small cell lung carcinoma (NSCLC), small cell lung cancer (SCLC), bladder cancer, prostate cancer (e.g., metastatic hormone-refractory prostate cancer), advanced breast cancer, advanced ovarian cancer, and/or advanced refractory solid tumor. In some embodiments, the combination treatment is designed for treating metastatic solid tumors, NSCLC, melanoma, B-cell non-Hodgkin lymphoma, colorectal cancer, and multiple myeloma. In some embodiments, the combination treatment is designed for treating melanoma, carcinomas (e.g., pancreatic carcinoma), mesothelioma, hematological malignancies (e.g., Non-Hodgkin's lymphoma, lymphoma, chronic lymphocytic leukemia), or solid tumors (e.g., advanced solid tumors).

In some embodiments, the methods described herein can be used to evaluate the combination treatment with some other methods. The methods of treating a cancer that can be used alone or in combination with methods described herein, include, e.g., treating the subject with chemotherapy, e.g., campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, and/or methotrexate. Alternatively or in addition, the methods can include performing surgery on the subject to remove at least a portion of the cancer, e.g., to remove a portion of or all of a tumor(s), from the subject.

### EXAMPLES

The invention is further described in the following examples, which do not limit the scope of the invention described in the claims.

### Materials and Methods

The following materials were used in the following examples.

C57BL/6 mice were purchased from the China Food and Drugs Research Institute National Rodent Experimental Animal Center.

EcoRI, BamHI, HindIII, EcoRV, KpnI restriction enzymes were purchased from NEB (Catalog numbers: R3101M, R3136M, R3104M, R0195S, R0142S).

Ambion in vitro transcription kit was purchased from Ambion (Catalog number: AM1354).

UCA kit was obtained from Beijing Biocytogen Co., Ltd. (Catalog number: BCG-DX-001).

Reverse Transcription Kit was purchased from Takara Bio, Inc. (Catalog number: 6110A).

E. coli TOP10 competent cells were purchased from Tiangen Biotech Co., Ltd. (Catalog number: CB104-02).

Cas9 mRNA was purchased from SIGMA (Catalog number: CAS9MRNA-1EA).

AIO kit was purchased from Beijing Biocytogen Co., Ltd. (Catalog number: BCG-DX-004).

pHSG299 plasmid was purchased from Takara Bio, Inc. (Catalog number: 3299).

Flow cytometer was purchased from BD Biosciences (model: FACS Calibur™)

### Example 1: Sequence Design for Caninized PD-1 Mice

PD-1 genes from non-human animals, such as mouse and dog, are usually transcribed into various isoforms. The sequence design in this example section is mainly illustrated using one of the isoforms. For example, a main part of exon 2 of the mouse PD-1 gene (Gene ID: 18566) was replaced by a corresponding fragment from the canine PD-1 gene (Gene ID: 486213).

The NCBI accession number for the mouse PD-1 gene and the protein is NM_008798.2→NP_032824.1. The mRNA sequence is shown in SEQ ID NO: 1, and the corresponding amino acid sequence is shown in SEQ ID NO: 2.

The NCBI accession number for the canine PD-1 gene and the protein is NM_001314097.1→NP_001301026.1. The mRNA sequence is shown in SEQ ID NO: 3, and the corresponding amino acid sequence is shown in SEQ ID NO: 4.

A schematic diagram that compares the mouse PD-1 gene and the canine PD-1 gene is shown in FIG. 1. A schematic diagram of the resulting genetically modified caninized mouse PD-1 gene is shown in FIG. 2. The DNA sequence of the caninized mouse PD-1 gene (chimeric PD-1 gene) is shown in SEQ ID NO: 5 as follows:

CCCCAATGGGccctggagcccgctcaccuctccccggcgcagctcacggt
gcaggagggagagaacgccacgttcacctgcagcctggccgacatccccg
acagcttcgtgctcaactggtaccgcctgagcccccgcaaccagacggac
aagctggccgccuccaggaggaccgcatcgagccgggccgggacaggcgc
ttccgcgtcaCgcggctgcccaacgggcgggacttccacatgagcatcgt
cgctgcgcgcctcaacgacagcggcatctacctgtgcggggccatctacc
tgccccccaacacacagatcaacgagagtccccgcgcagagCTCGTGGTA
A

SEQ ID NO: 5 only lists the DNA sequence involved in genetic modification, in which the underlined region is a fragment from the canine PD-1 gene.

The CDS region, the mRNA sequence and the encoded protein sequence of the genetically modified caninized mouse PD-1 are shown in SEQ ID NO: 6, SEQ ID NO: 7 and SEQ ID NO: 8, respectively.

### Example 2: Design and Construction of pClon-4G-DPD-1 Vector

Based on the sequence design, the inventors further designed a targeting strategy as shown in FIG. 3 and a vector comprising a 5′ homologous arm, the canine PD-1 gene fragment, and a 3′ homologous arm. Specifically, the 5′ homologous arm (SEQ ID NO: 9) comprises nucleic acid 94041502-94043271 of NCBI Accession No. NC_000067.6, and the 3′ homologous arm (SEQ ID NO: 10) comprises nucleic acid 94039436-94041168 of NCBI Accession No. NC_000067.6. The canine PD-1 gene fragment (SEQ ID NO: 11) has a mutation relative to nucleic acid 51611212-51611544 of NCBI Accession No. NC_006607.3, that is, a T at position 203 was mutated to C, and the mutation does not affect protein expression.

The vector was constructed as follows: upstream and corresponding downstream primers, as well as related sequences were designed to amplify the 5′ homologous arm and the 3′ homologous arm. Specifically, the 5′ homologous arm corresponds to the LR fragment, and the 3′ homologous arm corresponds to the RR fragment, and the primer sequences are as follows:

LR:
(SEQ ID NO: 12)
F: 5′-tttaagaaggagatatacatggctcgagtggcccatagagacca
atgtggac-3′
(SEQ ID NO: 13)
R: 5′-gagcgggctccagggcccattggggacctctgaaatgcag-3′
RR:
(SEQ ID NO: 16)
F: 5′-agtccccgcgcagagctcgtggtaacaggtgaggctagtag-3′
(SEQ ID NO: 17)
R: 5′-ttgttagcagccggatctcagtctagatgtgcacacaggcgg-3′

The LR and RR fragments were obtained by PCR amplification using C57BL/6 mouse DNA or BAC library as a template, and the canine gene fragment shown in SEQ ID NO: 11 was synthesized. The fragments was ligated by the AIO kit to the pClon-4G plasmid from the AIO kit to obtain the pClon-4G-DPD-1 vectors.

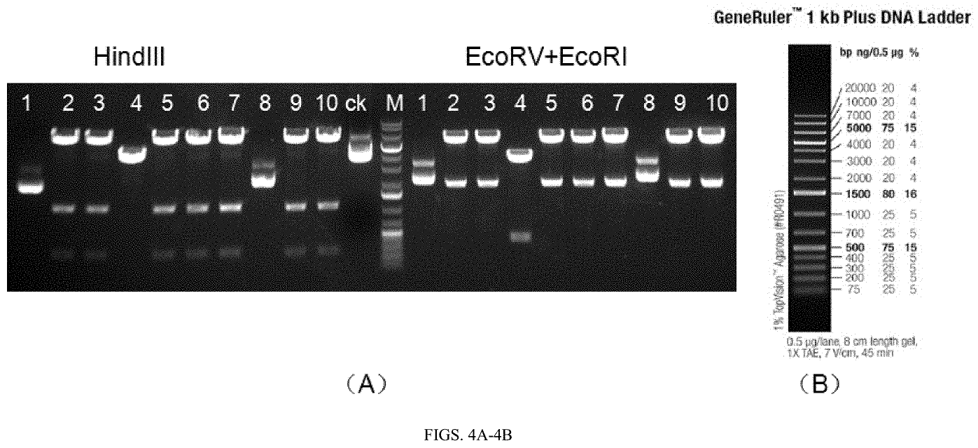

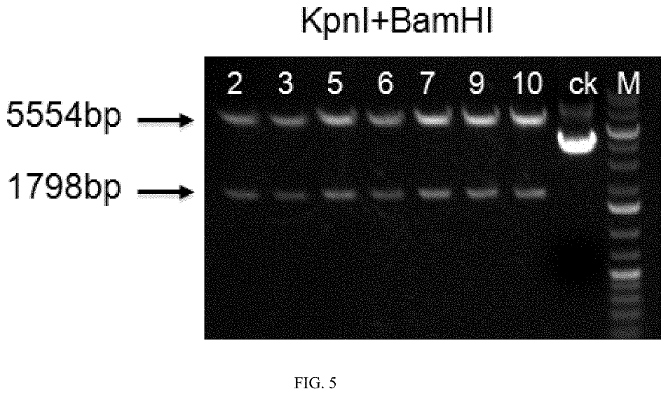

Ten pClon-4G-DPD-1 clones were randomly selected and verified by restriction endonuclease digestion. Among them, HindIII should generate 5984 bp+1098 bp+270 bp fragments, EcoRV+EcoRI should generate 5540 bp+1812 bp fragments, KpnI+BamHI should generate 5554 bp+1798 bp fragments. The results of restriction enzyme digestion are shown in FIGS. 4A-4B and 5. The digestion results of plasmid number 2, 3, 5, 6, 7, 9, and 10 were in agreement with the expected results, indicating that the plasmids had correct sequences. The sequences of Plasmids 3 and 5 were further verified by sequencing. Plasmid 3 was selected for subsequent experiments.

### Example 3: DESIGN and Screening of sgRNA Targeting PD-1 Gene

The target sequence determines the targeting specificity of sgRNAs and the efficiency of inducing Cas9 cleavage at the gene of interest. Thus, it is important to test the efficiency of the specific target sequence.

According to the targeting scheme, sgRNA sequences recognizing the 5′ end targeting site (sgRNA1-sgRNA4) and the 3′ end targeting site (sgRNA5-sgRNA8) were designed and synthesized.

Both the 5′ end targeting site and the 3′ end targeting site were located in exon 2 of the mouse PD-1 gene. The targeting site sequences on PD-1 for each sgRNA are shown below:

sgRNA1 target sequence (SEQ ID NO: 18):
5′-agggacctccagggcccattggg-3′
sgRNA2 target sequence (SEQ ID NO: 19):
5′-cagaggtccccaatgggccctgg-3′
sgRNA3 target sequence (SEQ ID NO: 20):
5′-gtagaaggtgagggacctccagg-3′
sgRNA4 target sequence (SEQ ID NO: 21):
5′-ccctcaccttctacccagcctgg-3′
sgRNA5 target sequence (SEQ ID NO: 22):
5′-gcaccccaaggcaaaaatcgagg-3′
sgRNA6 target sequence (SEQ ID NO: 23):
5′-ggagcagagctcgtggtaacagg-3′
sgRNA7 target sequence (SEQ ID NO: 24):
5′-gttaccacgagctctgctccagg-3′
sgRNA8 target sequence (SEQ ID NO: 25):
5′-gcaaaaatcgaggagagccctgg-3′

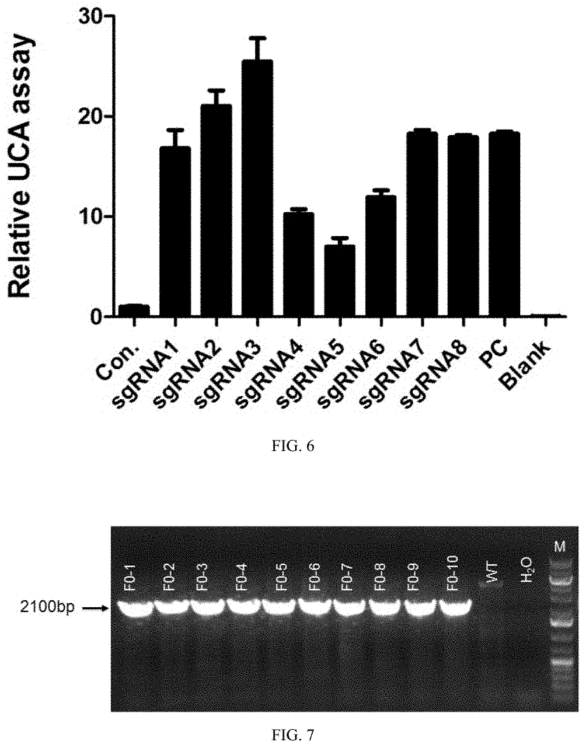

The UCA kit was used to detect the activities of sgRNAs. The results showed that the guide sgRNAs had different activities (see Table 3 and FIG. 6). The results of UCA showed that sgRNA-5 activity was the lowest in all targeting sites, and sgRNA-3 activity was the highest, which could be due to the specificity of the targeting site sequence. But according to the experiment, the value of sgRNA-5 activity was still significantly higher than that of the Con group activity. This indicated that sgRNA-5 was still active, and its activity would still be sufficient for the gene targeting experiment.

sgRNA-3 and sgRNA-8 were selected. A sequence of TAGG was added to the 5′ end of the upstream sequences to obtain a forward oligonucleotide, and a sequence of AAAC was added at the 5′ end of the complementary strand (downstream sequence) to obtain a reverse oligonucleotide. The synthesized forward and reverse oligonucleotides were used in subsequent experiments. The specific sequences are as follows:

sgRNA-3:
Upstream sequence:
(SEQ ID NO: 26)
5′-TAGAAGGTGAGGGACCTCC-3′
Forward oligonucleotide:
(SEQ ID NO: 27)
5′-TAGGTAGAAGGTGAGGGACCTCC-3′
Downstream sequence:
(SEQ ID NO: 28)
5′-GGAGGTCCCTCACCTTCTA-3′
Reverse oligonucleotide:
(SEQ ID NO: 29)
5′-AAACGGAGGTCCCTCACCTTCTA-3′
sgRNA-8:
Upstream sequence:
(SEQ ID NO: 30)
5′-CAAAAATCGAGGAGAGCCC-3′
Forward oligonucleotide:
(SEQ ID NO: 31)
5′-TAGGCAAAAATCGAGGAGAGCCC-3′
Downstream sequence:
(SEQ ID NO: 32)
5′-GGGCTCTCCTCGATTTTTG-3′
Reverse oligonucleotide:
(SEQ ID NO: 33)
5′-AAACGGGCTCTCCTCGATTTTTG-3′

TABLE 3
UCA test results

| Name | Relative Value |
|---|---|
| Con. | 1.0 ± 0.10 |
| sgRNA1 | 16.8 ± 0.11 |
| sgRNA2 | 21 ± 0.07 |
| sgRNA3 | 25.5 ± 0.09 |
| sgRNA4 | 10.2 ± 0.05 |
| sgRNA5 | 7.0 ± 0.12 |
| sgRNA6 | 11.9 ± 0.06 |
| sgRNA7 | 18.3 ± 0.02 |
| sgRNA8 | 17.9 ± 0.01 |
| PC | 18.3 ± 0.01 |
| Blank | 0.03 ± 0.03 |

### Example 4: pT7-sgRNA G2 Plasmid Construction

Plasmid pT7-sgRNA G2 was obtained as follows: A DNA fragment containing the T7 promoter and sgRNA scaffold (SEQ ID NO: 34) was synthesized by a plasmid synthesis company. The fragment was ligated into the backbone vector pHSG299 plasmid by restriction enzyme digestion (EcoRI and BamHI). The sequences were confirmed by sequencing.

The DNA fragment containing the T7 promoter and sgRNA scaffold (SEQ ID NO: 34):

Gaattctaatacgactcactatagggggtatcgagaagacctgttttaga
gctagaaatagcaagttaaaataaggctagtccgttatcaacttgaaaaa
gtggcaccgagtcggtgcttttaaaggatcc

### Example 5: Construction of Recombinant Expression Vector of pT7-sgRNA-DPD3 and pT7-sgRNA-DPD8

After annealing the forward and reverse oligonucleotides obtained in Example 3, the products were ligated to the pT7-sgRNA plasmid, respectively, to obtain expression vectors pT7-sgRNA-DPD3 and pT7-sgRNA-DPD8.

The following ligation reaction reagents (10 μL) were used: sgRNA annealing product, 1 μL (0.5 μM); pT7-sgRNA G2 vector, 1 μL (10 ng); T4 DNA Ligase, 1 μL (5 U); 10×T4 DNA Ligase buffer, 1 μL; 50% PEG 4000, 1 μL; H₂O, supplemented to a total volume of 10 μL.

The reaction conditions were as follows: ligation was performed at room temperature for 10-30 minutes, and the ligation product was used to transform 30 μL of TOP10 competent cells. Next, 200 μL of the transformed cells were plated onto a plate with Kanamycin, then incubated at 37° C. for at least 12 hours. Next, 2 clones were selected to inoculate LB culture (5 mL) with Kanamycin. The culture was incubated at 37° C. by shaking at 250 rpm for at least 12 hours.

Randomly selected clones were sequenced to verify sequences, and the correctly ligated expression vectors pT7-sgRNA-DPD3 and pT7-sgRNA-DPD8 were selected for subsequent experiments.

### Example 6: Microinjection and Embryo Transfer Using C57BL/6 Mice

The pre-mixed Cas9 mRNA, pClon-4G-DPD-1 plasmid and in vitro transcription products (using the Ambion in vitro transcription kit according to the protocols) of pT7-1c:1 sgRNA-DPD3, pT7-sgRNA-DPD8 plasmids were injected into the cytoplasm or nucleus of mouse fertilized eggs (C57BL/6 background) with a microinjection instrument. The embryo microinjection was carried out according to the method described, e.g., in A. Nagy, et al., “Manipulating the Mouse Embryo: A Laboratory Manual (Third Edition),” Cold Spring Harbor Laboratory Press, 2003. The injected fertilized eggs were then transferred to a culture medium for a short time culture, and then was transplanted into the oviduct of the recipient mouse to produce the genetically modified caninized mice (F0 generation). The mouse population was further expanded by cross-mating and self-mating to establish stable mouse lines.

### Example 7: Verification of Genetic Modified Caninized Mice

PCR analysis was performed using mouse tail genomic DNA of F0 generation mice. Primer L-GT-F is located on the left side of 5′ homologous arm, primer R-GT-R is located on the right side of 3′ homologous arm, and both Mut-R1 and Mut-F1 are located within the caninized gene fragment. The specific sequences are as follows:

5′ primers:
Upstream:
(SEQ ID NO: 35)
L-GT-F: 5′-CATCATACTGGCAACCCCTAGCCTG-3′
Downstream:
(SEQ ID NO: 36)
Mut-R1: 5′-GCTGTCGTTGAGGCGCGCAGCGAC-3′
3′ primers:
Upstream:
(SEQ ID NO: 37)
Mut-F1: 5′-CTGGCCGACATCCCCGACAGCTTCG-3′
Downstream:
(SEQ ID NO: 38)
L-GT-R: 5′-TGACAATAGGAAACCGGGAAGCCTG-3′

The reagents and the conditions for PCR are shown in the tables below.

TABLE 4
The PCR reaction (20 μL)

| | | |
|---|---|---|
| 2 × PCR buffer | 10 | μL |
| dNTP (2 μM) | 4 | μL |
| Upstream primer (10 μM) | 0.6 | μL |
| Downstream primer (10 μM) | 0.6 | μL |
| Mouse tail genomic DNA | 100 | ng |
| KOD-FX (1 U/μL) | 0.4 | μL |

| | |
|---|---|
| H₂O | Add to 20 μL |

TABLE 5
The PCR reaction conditions

| | | |
|---|---|---|
| Temperature | Time | Cycles |

| | | | |
|---|---|---|---|
| 94° C. | 2 | min | 1 |
| 98° C. | 10 | sec | 15 |
| 67° C. (−0.7° C./cycle) | 30 | sec | |
| 68° C. | 1 | kb/min | |
| 98° C. | 10 | sec | 25 |
| 56° C. | 30 | sec | |
| 68° C. | 1 | kb/min | |
| 68° C. | 10 | min | 1 |

| | | |
|---|---|---|
| 4° C. | as needed | 1 |

If the desired canine gene sequence was inserted into the correct positions in the genome, PCR experiments using the above primers should generate only one band. The 5′ end PCR experiment should produce a band of about 2100 bp, and the 3′ end PCR experiment should produce a band of about 2353 bp. The results for F0 generation mice are shown in FIGS. 7-8. Among them, all 10 mice labeled from F0-1 to F0-10 were positive.

### Example 8: Verification of Gene Knockout Mice

Because Cas9 cleavage can cause DNA double-strand breaks, and the repair by homologous recombination can result in insertion/deletion mutations, it is possible to obtain gene knockout mice that have lost function of mouse PD-1 protein while preparing the PD-1 gene caninized mice. Therefore, a pair of detecting primers were designed, which are located at the left side of the 5′ end targeting site and the right side of the 3′ end targeting site, respectively. The sequence is as follows:

(SEQ. ID. NO: 14)
KO-F: 5′-GGGAAGGTAGAGACATCTTCGGGGA-3′
(SEQ. ID. NO: 15)
KO-R: 5′-CGAGGGGCTGGGATATCTTGTTGAG-3′

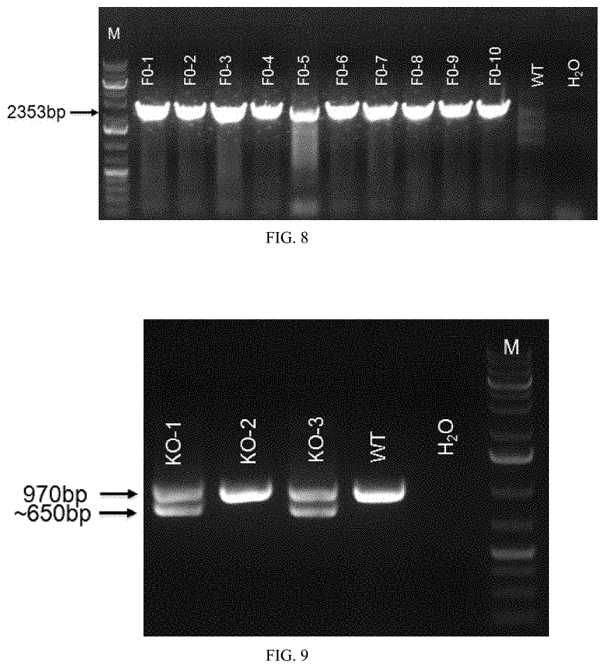

The wild-type mouse PCR product should be 970 bp in length and the knockout mouse product should be about 650 bp in length. The PCR results are shown in FIG. 9. Among the 3 tested mice, the mice labeled KO-1 and KO-3 were PD-1 gene knockout heterozygotes.

### Example 9: Preparation and Verification of Double-Caninized or Multi-Caninized Mice

Mice with the canine or chimeric PD-1 gene (e.g., animal model with dPD-1 prepared using the methods as described in the present disclosure) can also be used to prepare an animal model with double-caninized or multi-caninized genes. For example, in Example 6, the embryonic stem cell used in the microinjection and embryo transfer process can be selected from the embryos of other genetically modified mice, so as to obtain double- or multiple-gene modified mouse models. The fertilized eggs of mice with canine or chimeric PD-1 gene can also be further genetically engineered to produce mouse lines with one or more caninized or otherwise genetically modified mouse models. In addition, the genetically engineered PD-1 animal model homozygote or heterozygote can be mated with other genetically modified homozygous or heterozygous animal models (or through IVF), and the progeny can be screened. According to the Mendelian law, there is a chance to obtain the double-gene or multiple-gene modified heterozygous animals, and then the heterozygous animals can be further mated with each other to finally obtain the double-gene or multiple-gene modified homozygotes.

### Example 10. Pharmacological Validation of Caninized PD-1 Animal Model

Homozygous mice (4-6 weeks) with caninized PD-1 gene were subcutaneously injected with mouse colon cancer cell MC38, and when the tumor volume grew to about 100 mm³, the mice were divided to a control group and three treatment groups based on tumor size (n=5/group). In each treatment group, an anti-canine PD-1 monoclonal antibody (Ab1, Ab2, Ab3, obtained by using conventional methods to immunize mice, see Janeway's Immunobiology (9^{th} Edition)) was selected and injected to the mice at 10 mg/kg, while the control group was injected with an equal volume of saline solution. Intraperitoneal injection was performed and the frequency of administration was twice a week (6 times of administrations in total). The tumor volume was measured twice a week. Euthanasia was performed when the tumor volume of the mouse reached 3000 mm³.

Table 6 below shows results for this experiment, including the tumor volumes at the day of grouping (day 0), 18 days after the grouping (day 18), and at the end of the experiment (day 25), the survival rate of the mice, the tumor-free cases, and the Tumor Growth Inhibition value (TGI_{TV}) in the treatment and control groups.

TABLE 6
Tumor volume, survival rate and TGI_{TV}

| | | | | | | Tumor- | |
|---|---|---|---|---|---|---|---|
| | | Tumor Volume (mm³) | | | Survival | free | |
| | | Day 0 | Day 18 | Day 25 | rate | cases | TGI_{TV} % |
| Control group G1 | | 124 ± 4 | 1072 ± 248 | 2099 ± 551 | 5/5 | 0/5 | N/A |
| Treatment | G2 (Ab1) | 124 ± 12 | 691 ± 240 | 1402 ± 529 | 5/5 | 0/5 | 35.3% |
| group | G3 (Ab2) | 125 ± 12 | 451 ± 249 | 1021 ± 633 | 5/5 | 1/5 | 54.6% |
| | G4 (Ab3) | 124 ± 9 | 958 ± 283 | 2099 ± 686 | 5/5 | 0/5 | 0 |

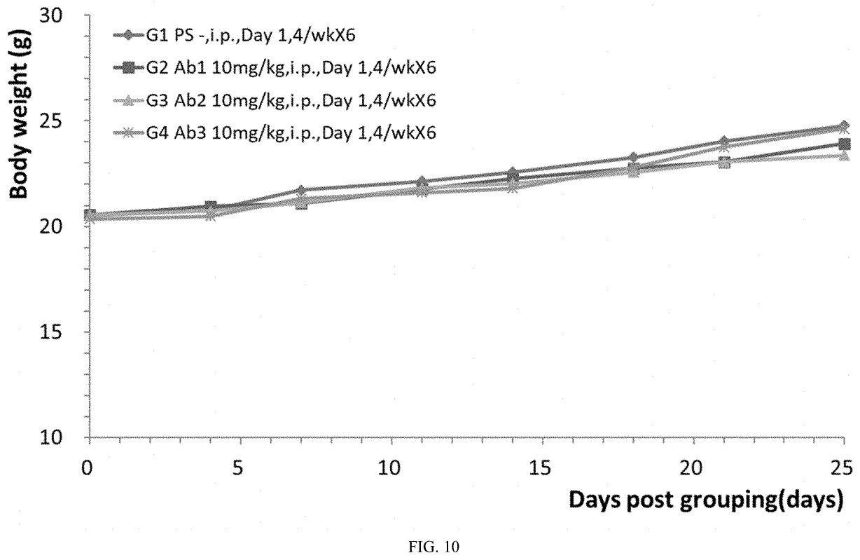

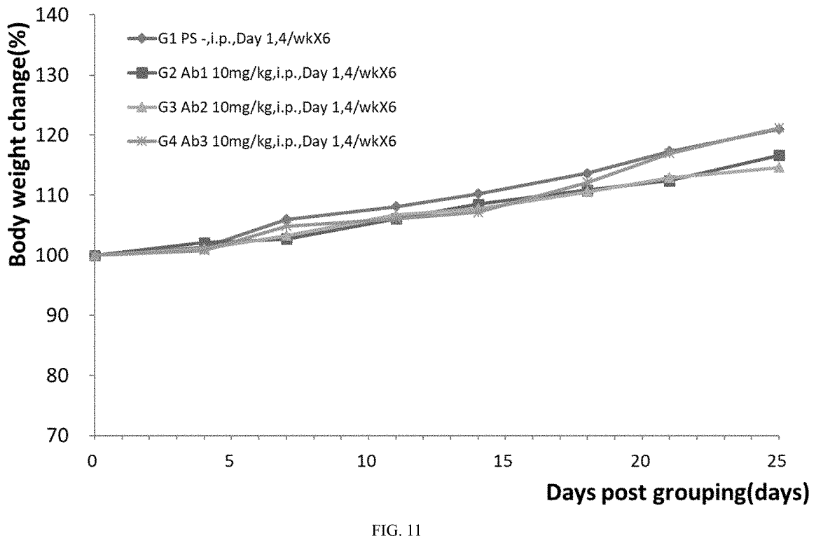

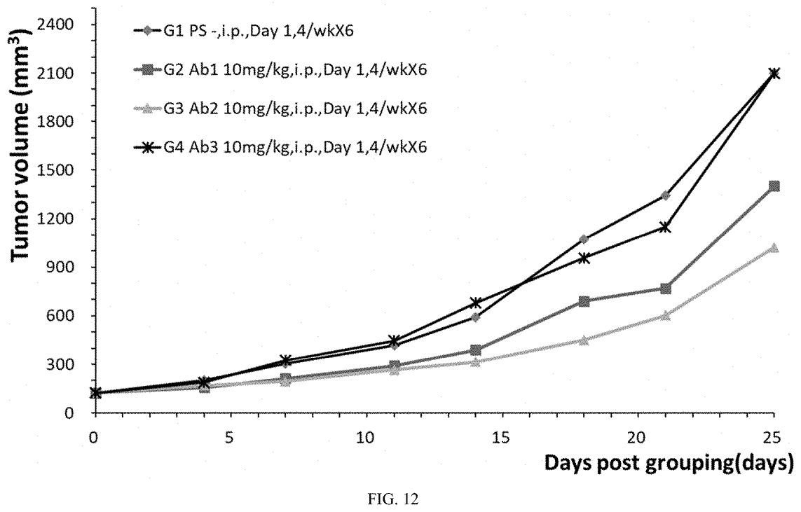

Overall, the animals in each group were healthy. At the end of the experiment, each group of animals had good weight gain (FIG. 10), and the body weight of each treatment group was not significantly different from the control group, indicating that the animals tolerated the 3 antibodies well. The mean weight gain changes of all treatment groups (G2-G4) and control group (G1) were not significantly different throughout the experimental period (FIG. 11), indicating that these three antibodies did not have significant toxic effects on animals, that is, these antibodies were relatively safe. In terms of therapeutic effect, the tumor volume difference was not significant between the antibody Ab3 (G4) treatment group and the control group (G1) (see FIG. 12). The average tumor volume of the mice in the antibody Ab1 (G2) and Ab2 (G3) treatment groups was 1402±529 mm³ and 1021±633 mm³, respectively. Compared with the control group (G1) (mean tumor volume was 2099±551 mm³), the tumor volume was significantly reduced, indicating these two anti-canine PD-1 monoclonal antibodies had an inhibitory effect against tumor growth, and antibody Ab2 was slightly better than antibody Ab1 in treating tumor.

In another experiment, homozygous mice (8-9 weeks) with caninized PD-1 gene were subcutaneously injected with mouse colon cancer cell MC38, which overexpressed PD-L1. When the tumor volume grew to about 300 mm³, the mice were divided to a control group and several treatment groups based on tumor size (n=7/group). For the treatment groups, an anti-canine PD-1 monoclonal antibody was selected with a treatment dosage of 0.3-10 mg/kg, while the control group was injected with an equal volume of saline solution. Intraperitoneal injection was performed and the frequency of administration was twice a week (6 times of administrations in total). The tumor volume was measured twice a week. Euthanasia was performed when the tumor volume of the mouse reached 3000 mm³.

Table 7 below shows results for this experiment, including the tumor volumes at the day of grouping (day 0), 17 days after the grouping (day 17), and at the end of the experiment (day 24), the survival rate of the mice, the tumor-free cases, the Tumor Growth Inhibition value (TGI_{TV}) in the treatment and control groups, and P value.

TABLE 7
Tumor volume, survival rate and TGI_{TV}

| | | | | | | Tumor- | | P value | |
|---|---|---|---|---|---|---|---|---|---|
| | | Tumor Volume (mm³) | | | Survival | free | | Body | Tumor |
| | | Day 0 | Day 17 | Day 24 | rate | cases | TGI_{TV} % | Weight | Volume |
| Control group G1 | | 322 ± 7 | 1763 ± 238 | 2017 ± 490 | 7/7 | 0/7 | N/A | N/A | N/A |
| Treat | G2 | 322 ± 9 | 155 ± 64 | 64 ± 54 | 7/7 | 5/7 | 115.2% | 0.252 | 0.002 |
| group | (10 mg/kg) | | | | | | | | |
| | G3 | 322 ± 8 | 238 ± 76 | 207 ± 85 | 7/7 | 2/7 | 106.8% | 0.137 | 0.003 |
| | (3 mg/kg) | | | | | | | | |
| | G4 | 322 ± 10 | 695 ± 454 | 816 ± 445 | 6/7 | 2/7 | 70.9 | 0.420 | 0.095 |
| | (0.3 mg/kg) | | | | | | | | |

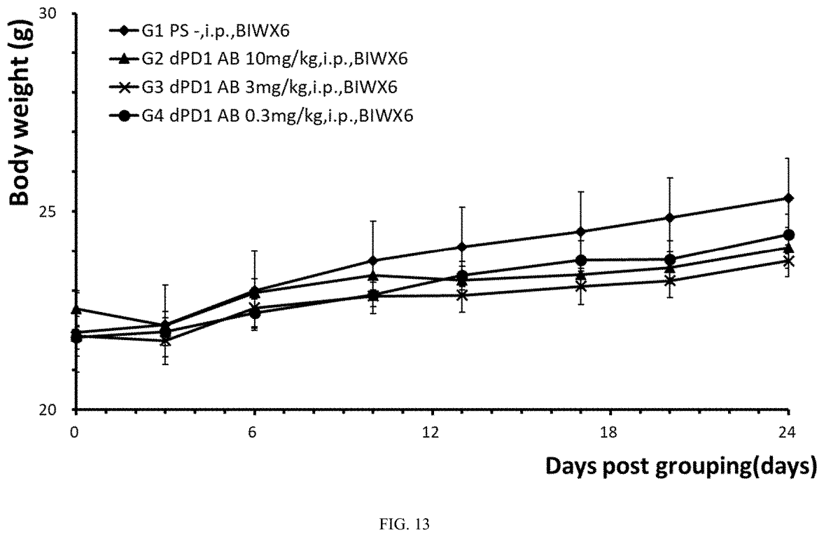

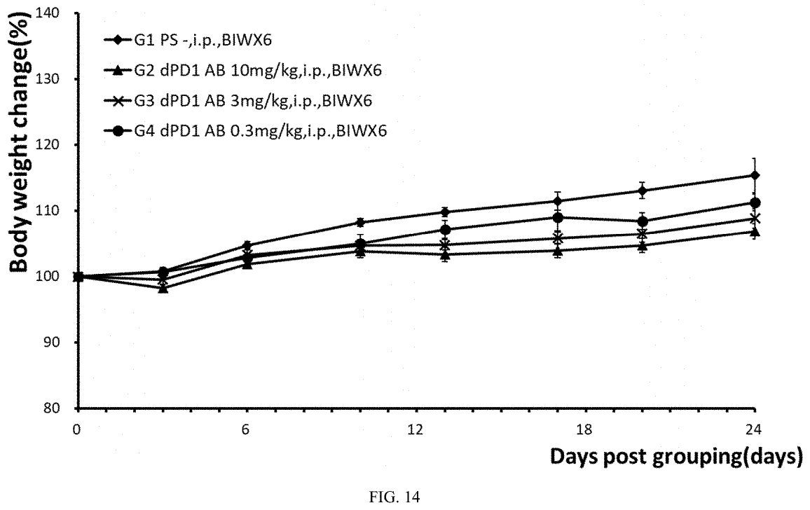

Overall, the animals in each group were healthy. At the end of the experiment, each group of animals had good weight gain (FIG. 13), and the body weight of each treatment group was not significantly different from the control group, indicating that the animals tolerated the anti-canine PD-1 monoclonal antibody well. The mean weight gain changes of all treatment groups (G2-G4) and control group (G1) were not significantly different throughout the experimental period (FIG. 14), indicating that the antibodies at the different treatment dosages did not have significant toxic effects on animals.

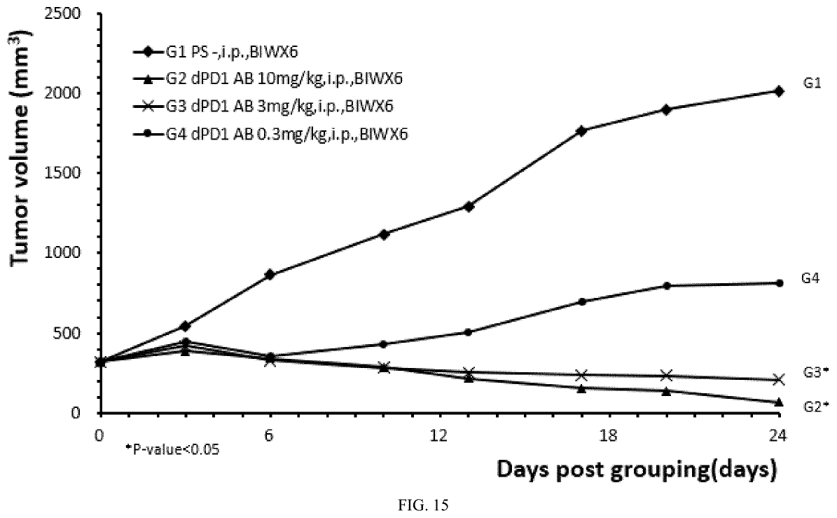

In the control group, tumors in all the mice continued to grow during the experiment, but among the 21 mice in all treatment groups, tumors in 9 mice disappeared at the end of the experiment (FIG. 15). At the end of the experiment, the mean tumor volume of the control group was 2017±490 mm³, and the mean tumor volume of the treatment groups with treatment dosages of 10 mg/kg, 3 mg/kg, and 0.3 mg/kg were 64±54 mm³, 207±85 mm³, and 816±445 mm³, respectively. The tumor volumes of all treatment group mice were significantly smaller than that of the control group mice. TGI_{TV} of each treatment group was determined as 115.2%, 106.8%, and 70.9%, respectively, indicating that different doses of PD-1 monoclonal antibody had significant tumor inhibition effects (TGI_{TV}>60%), and the therapeutic effect is correlated with the dosage.

The above experiments demonstrated that the caninized PD-1 gene-modified mice generated by the method can be used for screening PD-1 targeting antibodies and in vivo pharmacological tests. Also, the caninized PD-1 mice can be used as a living replacement model for in vivo studies of the canine PD-1 signaling pathway modulator screening, assessment and treatment.

### Example 11. Methods Based on Embryonic Stem Cell Technologies

The mammals (e.g., non-canine, non-human mammals) described herein can also be prepared through other gene editing systems and approaches, including but not limited to: gene homologous recombination techniques based on embryonic stem cells (ES), zinc finger nuclease (ZFN) techniques, transcriptional activator-like effector factor nuclease (TALEN) technique, homing endonuclease (megakable base ribozyme), or other techniques.

Since the goal is to replace exon 2 of the mouse PD-1 gene in whole or in part with the canine PD-1 gene, a recombinant vector that contains a 5′ homologous arm, a 3′ homologous arm, and a sequence fragment from canine PD-1 is designed. The vector can also contain a resistance gene for positive clone screening, such as neomycin phosphotransferase coding sequence Neo. On both sides of the resistance gene, two site-specific recombination systems in the same orientation, such as Frt or LoxP, can be added. Furthermore, a coding gene with a negative screening marker, such as the diphtheria toxin A subunit coding gene (DTA), can be constructed downstream of the recombinant vector 3′ homologous arm.

Vector construction can be carried out using methods known in the art, such as enzyme digestion and so on. The recombinant vector with correct sequence can be next transfected into mouse embryonic stem cells, such as C57BL/6 mouse embryonic stem cells, and then the recombinant vector can be screened by positive clone screening gene. The cells transfected with the recombinant vector are next screened by using the positive clone marker gene, and Southern Blot technique can be used for DNA recombination identification. For the selected correct positive clones, the positive clonal cells (black mice) are injected into the isolated blastocysts (white mice) by microinjection according to the method described in the book A. Nagy, et al., “Manipulating the Mouse Embryo: A Laboratory Manual (Third Edition),” Cold Spring Harbor Laboratory Press, 2003. The resulting chimeric blastocysts formed following the injection are transferred to the culture medium for a short time culture and then transplanted into the fallopian tubes of the recipient mice (white mice) to produce F0 generation chimeric mice (black and white). The F0 generation chimeric mice with correct gene recombination are then selected by extracting the mouse tail genome and detecting by PCR for subsequent breeding and identification. The F1 generation mice are obtained by mating the F0 generation chimeric mice with wild-type mice. Stable gene recombination positive F1 heterozygous mice are selected by extracting mouse tail genome and PCR detection. Next, the F1 heterozygous mice are mated to each other to obtain genetically recombinant positive F2 generation homozygous mice. In addition, the F1 heterozygous mice can also be mated with F1p or Cre mice to remove the positive clone screening marker gene (e.g., Neo), and then the caninized PD-1 gene homozygous mice can be obtained by mating these mice with each other. The methods of genotyping and using the F1 heterozygous mice or F2 homozygous mice are similar to the methods as described in the early Examples. The results showed that mice with caninized PD-1 gene can also be prepared by using gene homologous recombination techniques based on ES cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically-modified, non-human, non-canine animal whose genome comprises at least one chromosome comprising a sequence encoding a canine or chimeric PD-1.

2. The animal of claim 1, wherein the sequence encoding the canine or chimeric PD-1 is operably linked to an endogenous regulatory element at the endogenous PD-1 gene locus in the at least one chromosome.

3. The animal of claim 1, wherein the sequence encoding a canine or chimeric PD-1 comprises a sequence encoding an amino acid sequence that is at least 70%, 75%, 80%, 85%, 90%, 95%, 99%, or 100% identical to canine PD-1 (NP_001301026.1 (SEQ ID NO: 4)).

4. The animal of claim 1, wherein the sequence encoding a canine or chimeric PD-1 comprises a sequence encoding an amino acid sequence that is at least 70%, 75%, 80%, 85%, 90%, 95%, 99%, or 100% identical to SEQ ID NO: 8.

5. The animal of any one of claims 1-4, wherein the animal is a mammal, e.g., a monkey, a rodent or a mouse.

6. The animal of any one of claims 1-4, wherein the animal is a mouse or a rat.

7. The animal of any one of claims 1-6, wherein the animal does not express endogenous PD-1.

8. The animal of claim 1, wherein the animal has one or more cells expressing canine or chimeric PD-1.

9. The animal of claim 1, wherein the animal has one or more cells expressing canine or chimeric PD-1, and canine PD-L1 or canine PD-L2 can bind to the expressed canine or chimeric PD-1.

10. The animal of claim 1, wherein the animal has one or more cells expressing canine or chimeric PD-1, and endogenous PD-L1 or endogenous PD-L2 can bind to the expressed canine or chimeric PD-1.

11. A genetically-modified, non-human, non-canine animal, wherein the genome of the animal comprises a replacement of a sequence encoding a region of endogenous PD-1 with a sequence encoding a canine PD-1 or a chimeric PD-1 at an endogenous PD-1 gene locus.

12. The animal of claim 11, wherein the sequence encoding the canine PD-1 or the chimeric PD-1 is operably linked to an endogenous regulatory element at the endogenous PD-1 locus, and one or more cells of the animal express the canine PD-1 or the chimeric PD-1.

13. The animal of claim 11, wherein the animal does not express endogenous PD-1.

14. The animal of claim 11, wherein the replaced locus is located after start codon at the endogenous PD-1 locus.

15. The animal of claim 11, wherein the animal has one or more cells expressing a chimeric PD-1 having an extracellular region, a transmembrane region, and a cytoplasmic region, wherein the extracellular region comprises a sequence that is at least 50%, 60%, 70%, 80%, 90%, 95%, or 99% identical to the extracellular region of canine PD-1.

16. The animal of claim 15, wherein the extracellular region of the chimeric PD-1 has a sequence that has at least 10, 20, 30, 40, 50, 60, 70, 80, 90, or 100 contiguous amino acids that are identical to a contiguous sequence present in the extracellular region of canine PD-1.

17. The animal of claim 11, wherein the animal is a mouse, and the replaced region is in exon 2 of the endogenous mouse PD-1 gene.

18. The animal of claim 11, wherein the animal is heterozygous with respect to the replacement at the endogenous PD-1 gene locus.

19. The animal of claim 11, wherein the animal is homozygous with respect to the replacement at the endogenous PD-1 gene locus.

20. A method for making a genetically-modified, non-human, non-canine animal, comprising:
replacing in at least one cell of the animal, at an endogenous PD-1 gene locus, a sequence encoding a region of an endogenous PD-1 with a sequence comprising at least one exon of canine PD-1 gene or at least one chimeric exon (e.g., canine/mouse chimeric exon).

21. The method of claim 20, wherein the sequence comprising at least one exon of canine PD-1 gene comprises exon 1, exon 2, exon 3, exon 4, and/or exon 5, or a part thereof, of a canine PD-1 gene.

22. The method of claim 20, wherein the sequence comprising at least one exon of canine PD-1 gene comprises exon 1, exon 2, and/or exon 3, or a part thereof, of a canine PD-1 gene.

23. The method of claim 20, wherein the sequence comprising at least a sequence encoding at least amino acids 31-141 of SEQ ID NO: 4.

24. The method of claim 20, wherein the animal is a mouse, and the endogenous PD-1 gene locus is located at exon 1, exon 2, exon 3, exon 4, and/or exon 5 of the mouse PD-1 gene.

25. The method of claim 20, wherein the region is located in exon 2 of the mouse PD-1 gene, wherein the entire exon 2 or part of exon 2 is replaced with canine PD-1.

26. A non-human animal comprising at least one cell comprising a nucleotide sequence encoding a chimeric PD-1 polypeptide, wherein the chimeric PD-1 polypeptide comprises at least 50 contiguous amino acid residues that are identical to the corresponding contiguous amino acid sequence of a canine PD-1, wherein the animal expresses the chimeric PD-1.

27. The animal of claim 26, wherein the chimeric PD-1 polypeptide has at least 50 contiguous amino acid residues that are identical to the corresponding contiguous amino acid sequence of a canine PD-1 extracellular region.

28. The animal of claim 26, wherein the chimeric PD-1 polypeptide comprises a sequence that is at least 90%, 95%, or 99% identical to amino acids 31-141 of SEQ ID NO: 4.

29. The animal of claim 26, wherein the nucleotide sequence is operably linked to an endogenous PD-1 regulatory element of the animal.

30. The animal of claim 26, wherein the chimeric PD-1 polypeptide comprises an endogenous PD-1 transmembrane region and/or an endogenous PD-1 cytoplasmic region.

31. The animal of claim 26, wherein the nucleotide sequence is integrated to an endogenous PD-1 gene locus of the animal.

32. The animal of claim 26, wherein the chimeric PD-1 has at least one mouse PD-1 activity and/or at least one canine PD-1 activity.

33. A method of making a genetically-modified mouse cell that expresses a canine PD-1 or a chimeric PD-1, the method comprising:
replacing at an endogenous mouse PD-1 gene locus, a nucleotide sequence encoding a region of mouse PD-1 with a nucleotide sequence encoding a canine PD-1 or a chimeric PD-1, thereby generating a genetically-modified mouse cell that includes a nucleotide sequence that encodes the canine PD-1 or the chimeric PD-1, wherein the mouse cell expresses the canine PD-1 or the chimeric PD-1.

34. The method of claim 33, wherein the chimeric PD-1 comprises:
an extracellular region of canine PD-1; and
a transmembrane and/or a cytoplasmic region of mouse PD-1.

35. The method of claim 33, wherein the nucleotide sequence encoding the canine PD-1 or the chimeric PD-1 is operably linked to an endogenous PD-1 regulatory region, e.g., a promoter.

36. The animal of any one of claims 1-19 and 26-32, wherein the animal further comprises a sequence encoding an additional canine or chimeric protein.

37. The animal of claim 37, wherein the additional canine or chimeric protein is cytotoxic T-lymphocyte-associated protein 4 (CTLA-4), Lymphocyte Activating 3 (LAG-3), B And T Lymphocyte Associated (BTLA), Programmed Cell Death 1 Ligand 1 (PD-L1), CD3, CD27, CD28, CD40, CD47, CD137, CD154, T-Cell Immunoreceptor With Ig And ITIM Domains (TIGIT), T-cell Immunoglobulin and Mucin-Domain Containing-3 (TIM-3), Glucocorticoid-Induced TNFR-Related Protein (GITR), SIRPA (Signal Regulatory Protein Alpha), or TNF Receptor Superfamily Member 4 (OX40).

38. The method of any one of claims 20-25 and 33-35, wherein the animal or mouse further comprises a sequence encoding an additional canine or chimeric protein.

39. The method of claim 38, wherein the additional canine or chimeric protein is CTLA-4, LAG-3, BTLA, PD-L1, CD3, CD3e, CD27, CD28, CD40, CD47, CD137, CD154, SIPRA, TIGIT, TIM-3, GITR, or OX40.

40. A method of determining effectiveness of an anti-PD-1 antibody for the treatment of cancer, comprising:
administering the anti-PD-1 antibody to the animal of any one of claims 1-20 and 27-33, wherein the animal has a tumor; and
determining the inhibitory effects of the anti-PD-1 antibody to the tumor.

41. The method of claim 40, wherein the tumor comprises one or more cells that express a PD-1 ligand.

42. The method of claim 40, wherein the tumor comprises one or more cancer cells that are injected into the animal.

43. The method of claim 40, wherein determining the inhibitory effects of the anti-PD-1 antibody to the tumor comprises measuring the tumor volume in the animal.

44. The method of claim 40, wherein the tumor cells are melanoma cells, pancreatic carcinoma cells, mesothelioma cells, or solid tumor cells.

45. A method of determining effectiveness of an anti-PD-1 antibody and an additional therapeutic agent for the treatment of a tumor, comprising
administering the anti-PD-1 antibody and the additional therapeutic agent to the animal of any one of claims 1-20 and 27-33, wherein the animal has a tumor; and
determining the inhibitory effects on the tumor.

46. The method of claim 45, wherein the animal further comprises a sequence encoding a canine or chimeric CTLA4.

47. The method of claim 45, wherein the animal further comprises a sequence encoding a canine or chimeric programmed death-ligand 1 (PD-L1).

48. The method of claim 45, wherein the additional therapeutic agent is an anti-PD-L1 antibody or an anti-CTLA4 antibody.

49. The method of claim 45, wherein the tumor comprises one or more tumor cells that express PD-L1 or PD-L2.

50. The method of claim 45, wherein the tumor is caused by injection of one or more cancer cells into the animal.

51. The method of claim 45, wherein determining the inhibitory effects of the treatment involves measuring the tumor volume in the animal.

52. The method of claim 45, wherein the animal has melanoma, pancreatic carcinoma, mesothelioma, hematological malignancies (e.g., Non-Hodgkin's lymphoma, lymphoma, chronic lymphocytic leukemia), or solid tumors.

53. A protein comprising an amino acid sequence, wherein the amino acid sequence is one of the following:
(a) an amino acid sequence set forth in SEQ ID NO: 8;
(b) an amino acid sequence that is at least 90% identical to SEQ ID NO: 8;
(c) an amino acid sequence that is at least 91%, 92%, 93%, 94%, 95%, 96%, 97%, 98%, or 99% identical to SEQ ID NO: 8;
(d) an amino acid sequence that is different from the amino acid sequence set forth in SEQ ID NO: 8 by no more than 10, 9, 8, 7, 6, 5, 4, 3, 2 or 1 amino acid; and
(e) an amino acid sequence that comprises a substitution, a deletion and/or insertion of one, two, three, four, five or more amino acids to the amino acid sequence set forth in SEQ ID NO: 8.

54. A nucleic acid comprising a nucleotide sequence, wherein the nucleotide sequence is one of the following:
(a) a sequence that encodes the protein of claim 53;
(b) SEQ ID NO: 5, SEQ ID NO: 6, or SEQ ID NO: 7;
(c) a sequence that is at least 90% identical to SEQ ID NO: 5, SEQ ID NO: 6, or SEQ ID NO: 7; and
(d) a sequence that is at least 91%, 92%, 93%, 94%, 95%, 96%, 97%, 98%, or 99% identical to SEQ ID NO: 5, SEQ ID NO: 6, or SEQ ID NO: 7.

55. A cell comprising the protein of claim 53 and/or the nucleic acid of claim 54.

56. An animal comprising the protein of claim 53 and/or the nucleic acid of claim 54.

57. A method of determining effectiveness of an anti-PD-L1 antibody for the treatment of cancer, comprising:
administering the anti-PD-L1 antibody to the animal of any one of claims 1-20 and 27-33, wherein the animal has a tumor; and
determining the inhibitory effects of the anti-PD-L1 antibody to the tumor.

58. The method of claim 57, wherein the tumor comprises one or more cells that express PD-L1.

59. The method of claim 57, wherein the tumor comprises one or more cancer cells that are injected into the animal.

60. The method of claim 57, wherein determining the inhibitory effects of the anti-PD-L1 antibody to the tumor comprises measuring the tumor volume in the animal.


SEQUENCE LISTING (38 sequences)

SEQ ID NO: 1 - DNA, 1972 bp, Mus musculus
tgagcagcggggaggaggaagaggagactgctactgaaggcgacactgccaggggctctgggcatgtgggtccggcaggtaccctggtcattcacttgggctgtgctgcagttgagctggcaatcagggtggcttctagaggtccccaatgggccctggaggtccctcaccttctacccagcctggctcacagtgtcagagggagcaaatgccaccttcacctgcagcttgtccaactggtcggaggatcttatgctgaactggaaccgcctgagtcccagcaaccagactgaaaaacaggccgccttctgtaatggtttgagccaacccgtccaggatgcccgcttccagatcatacagctgcccaacaggcatgacttccacatgaacatccttgacacacggcgcaatgacagtggcatctacctctgtggggccatctccctgcaccccaaggcaaaaatcgaggagagccctggagcagagctcgtggtaacagagagaatcctggagacctcaacaagatatcccagcccctcgcccaaaccagaaggccggtttcaaggcatggtcattggtatcatgagtgccctagtgggtatccctgtattgctgctgctggcctgggccctagctgtcttctgctcaacaagtatgtcagaggccagaggagctggaagcaaggacgacactctgaaggaggagccttcagcagcacctgtccctagtgtggcctatgaggagctggacttccagggacgagagaagacaccagagctccctaccgcctgtgtgcacacagaatatgccaccattgtcttcactgaagggctgggtgcctcggccatgggacgtaggggctcagctgatggcctgcagggtcctcggcctccaagacatgaggatggacattgttcttggcctctttgaccagattcttcagccattagcatgctgcagaccctccacagagagcaccggtccgtccctcagtcaagaggagcatgcaggctacagttcagccaaggctcccagggtctgagctagctggagtgacagcccagcgcctgcaccaattccagcacatgcactgttgagtgagagctcacttcaggtttaccacaagctgggagcagcaggcttcccggtttcctattgtcacaaggtgcagagctggggcctaagcctatgtctcctgaatcctactgttgggcacttctagggacttgagacactatagccaatggcctctgtgggttctgtgcctggaaatggagagatctgagtacagcctgctttgaatggccctgtgaggcaaccccaaagcaagggggtccaggtatactatgggcccagcacctaaagccacccttgggagatgatactcaggtgggaaattcgtagactgggggactgaaccaatcccaagatctggaaaagttttgatgaagacttgaaaagctcctagcttcgggggtctgggaagcatgagcacttaccaggcaaaagctccgtgagcgtatctgctgtccttctgcatgcccaggtacctcagtttttttcaacagcaaggaaactagggcaataaagggaaccagcagagctagagccacccacacatccagggggcacttgactctccctactcctcctaggaaccaaaaggacaaagtccatgttgacagcagggaaggaaagggggatataaccttgacgcaaaccaacactggggtgttagaatctcctcattcactctgtcctggagttgggttctggctctccttcacacctaggactctgaaatgagcaagcacttcagacagtcagggtagcaagagtctagctgtctggtgggcacccaaaatgaccagggcttaagtccctttcctttggtttaagcccgttataattaaatggtaccaaaagctttaaaaaaaaaaaaaaaaaaaaaaaaaaaaaaaaaaaaaaaaaa

SEQ ID NO: 2 - protein, 288 aa, Mus musculus
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 3 - DNA, 942 bp, Canis familiaris
gcgggagccgccgggggaggcgagcaggcgggctggcgctccgggcatggggagccggcgggggccctggccgctcgtctgggccgtgctgcagctgggctggtggccaggatggctcctagactcccctgacaggccctggagcccgctcaccttctccccggcgcagctcacggtgcaggagggagagaacgccacgttcacctgcagcctggccgacatccccgacagcttcgtgctcaactggtaccgcctgagcccccgcaaccagacggacaagctggccgccttccaggaggaccgcatcgagccgggccgggacaggcgcttccgcgtcacgcggctgcccaacgggcgggacttccacatgagcatcgtcgctgcgcgcctcaacgacagcggcatctacctgtgcggggccatctacctgccccccaacacacagatcaacgagagtccccgcgcagagctctccgtgacggagagaaccctggagccccccacacagagccccagccccccacccagactcagcggccagttgcaggggctggtcatcggcgtcacgagcgtgctggtgggtgtcctgctactgctgctgctgacctgggtcctggccgctgtcttccccagggccacccgaggtgcctgtgtgtgcgggagcgaggacgagcctctgaaggagggccccgatgcagcgcccgtcttcaccctggactacggggagctggacttccagtggcgagagaagacgccggagcccccggcgccctgtgccccggagcagaccgagtatgccaccatcgtcttcccgggcaggccggcgtccccgggccgcagggcctcggccagcagcctgcagggagcccagcctccgagccccgaggacggacccggcctgtggcccccctgaccggccgcctccgctggcccatgtcctgc

SEQ ID NO: 4 - protein, 288 aa, Canis familiaris
MGSRRGPWPLVWAVLQLGWWPGWLLDSPDRPWSPLTFSPAQLTVQEGENATFTCSLADIPDSFVLNWYRLSPRNQTDKLAAFQEDRIEPGRDRRFRVTRLPNGRDFHMSIVAARLNDSGIYLCGAIYLPPNTQINESPRAELSVTERTLEPPTQSPSPPPRLSGQLQGLVIGVTSVLVGVLLLLLLTWVLAAVFPRATRGACVCGSEDEPLKEGPDAAPVFTLDYGELDFQWREKTPEPPAPCAPEQTEYATIVFPGRPASPGRRASASSLQGAQPPSPEDGPGLWPP

SEQ ID NO: 5 - DNA, 353 bp, Artificial Sequence
  DNA sequence of caninized mouse PD-1 gene
ccccaatgggccctggagcccgctcaccttctccccggcgcagctcacggtgcaggagggagagaacgccacgttcacctgcagcctggccgacatccccgacagcttcgtgctcaactggtaccgcctgagcccccgcaaccagacggacaagctggccgccttccaggaggaccgcatcgagccgggccgggacaggcgcttccgcgtcacgcggctgcccaacgggcgggacttccacatgagcatcgtcgctgcgcgcctcaacgacagcggcatctacctgtgcggggccatctacctgccccccaacacacagatcaacgagagtccccgcgcagagctcgtggtaa

SEQ ID NO: 6 - DNA, 867 bp, Artificial Sequence
  CDS region of caninized mouse PD-1
atgtgggtccggcaggtaccctggtcattcacttgggctgtgctgcagttgagctggcaatcagggtggcttctagaggtccccaatgggccctggagcccgctcaccttctccccggcgcagctcacggtgcaggagggagagaacgccacgttcacctgcagcctggccgacatccccgacagcttcgtgctcaactggtaccgcctgagcccccgcaaccagacggacaagctggccgccttccaggaggaccgcatcgagccgggccgggacaggcgcttccgcgtcacgcggctgcccaacgggcgggacttccacatgagcatcgtcgctgcgcgcctcaacgacagcggcatctacctgtgcggggccatctacctgccccccaacacacagatcaacgagagtccccgcgcagagctcgtggtaacagagagaatcctggagacctcaacaagatatcccagcccctcgcccaaaccagaaggccggtttcaaggcatggtcattggtatcatgagtgccctagtgggtatccctgtattgctgctgctggcctgggccctagctgtcttctgctcaacaagtatgtcagaggccagaggagctggaagcaaggacgacactctgaaggaggagccttcagcagcacctgtccctagtgtggcctatgaggagctggacttccagggacgagagaagacaccagagctccctaccgcctgtgtgcacacagaatatgccaccattgtcttcactgaagggctgggtgcctcggccatgggacgtaggggctcagctgatggcctgcagggtcctcggcctccaagacatgaggatggacattgttcttggcctctttga

SEQ ID NO: 7 - DNA, 1972 bp, Artificial Sequence
  mRNA sequence of  caninized mouse PD-1
tgagcagcggggaggaggaagaggagactgctactgaaggcgacactgccaggggctctgggcatgtgggtccggcaggtaccctggtcattcacttgggctgtgctgcagttgagctggcaatcagggtggcttctagaggtccccaatgggccctggagcccgctcaccttctccccggcgcagctcacggtgcaggagggagagaacgccacgttcacctgcagcctggccgacatccccgacagcttcgtgctcaactggtaccgcctgagcccccgcaaccagacggacaagctggccgccttccaggaggaccgcatcgagccgggccgggacaggcgcttccgcgtcacgcggctgcccaacgggcgggacttccacatgagcatcgtcgctgcgcgcctcaacgacagcggcatctacctgtgcggggccatctacctgccccccaacacacagatcaacgagagtccccgcgcagagctcgtggtaacagagagaatcctggagacctcaacaagatatcccagcccctcgcccaaaccagaaggccggtttcaaggcatggtcattggtatcatgagtgccctagtgggtatccctgtattgctgctgctggcctgggccctagctgtcttctgctcaacaagtatgtcagaggccagaggagctggaagcaaggacgacactctgaaggaggagccttcagcagcacctgtccctagtgtggcctatgaggagctggacttccagggacgagagaagacaccagagctccctaccgcctgtgtgcacacagaatatgccaccattgtcttcactgaagggctgggtgcctcggccatgggacgtaggggctcagctgatggcctgcagggtcctcggcctccaagacatgaggatggacattgttcttggcctctttgaccagattcttcagccattagcatgctgcagaccctccacagagagcaccggtccgtccctcagtcaagaggagcatgcaggctacagttcagccaaggctcccagggtctgagctagctggagtgacagcccagcgcctgcaccaattccagcacatgcactgttgagtgagagctcacttcaggtttaccacaagctgggagcagcaggcttcccggtttcctattgtcacaaggtgcagagctggggcctaagcctatgtctcctgaatcctactgttgggcacttctagggacttgagacactatagccaatggcctctgtgggttctgtgcctggaaatggagagatctgagtacagcctgctttgaatggccctgtgaggcaaccccaaagcaagggggtccaggtatactatgggcccagcacctaaagccacccttgggagatgatactcaggtgggaaattcgtagactgggggactgaaccaatcccaagatctggaaaagttttgatgaagacttgaaaagctcctagcttcgggggtctgggaagcatgagcacttaccaggcaaaagctccgtgagcgtatctgctgtccttctgcatgcccaggtacctcagtttttttcaacagcaaggaaactagggcaataaagggaaccagcagagctagagccacccacacatccagggggcacttgactctccctactcctcctaggaaccaaaaggacaaagtccatgttgacagcagggaaggaaagggggatataaccttgacgcaaaccaacactggggtgttagaatctcctcattcactctgtcctggagttgggttctggctctccttcacacctaggactctgaaatgagcaagcacttcagacagtcagggtagcaagagtctagctgtctggtgggcacccaaaatgaccagggcttaagtccctttcctttggtttaagcccgttataattaaatggtaccaaaagctttaaaaaaaaaaaaaaaaaaaaaaaaaaaaaaaaaaaaaaaaaa

SEQ ID NO: 8 - protein, 288 aa, Artificial Sequence
  Protein sequence  of caninized mouse PD-1
MWVRQVPWSFTWAVLQLSWQSGWLLEVPNGPWSPLTFSPAQLTVQEGENATFTCSLADIPDSFVLNWYRLSPRNQTDKLAAFQEDRIEPGRDRRFRVTRLPNGRDFHMSIVAARLNDSGIYLCGAIYLPPNTQINESPRAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 9 - DNA, 1770 bp, Artificial Sequence
  5′ homologous arm
tggcccatagagaccaatgtggaccactgctcaccctgcccagaaccatatgacccagtcttcaccaactctgcatggaatctagggatcctggcccttgaggagcgccagacccagctcataggccacgcccaccctcaggtctaagtgacattagattatctgtatgttcatcatccatggtacagctgagaacatcaaggagggaaagtggcagtcctacttttcgccatgtggtgatggagaccatttctgggcaaggctacatggtgcggatggatgtctctgggtttgcctctgctgaagtctgcttgctcactgcagacagctctgccacgtatctctggcttcctttctgcgctggaagatttcacataccttgtttgccaggtgttttgggcctcagttctccccccatccagcttctccctaactggcccctcttctttgcctctgacccctgctttctgagcccataaccttagctgtggcagcacagcctctctctttgtacccctgggagggaaccatgcccggttagtattgtcaaataccccacatcagaggcgggtgtgaggtttggggtgcagtgccctgggccatgtaatcgggtagaattccctccctatatgactactcaatccgtgggaggagagggcagagggctggaaaggatgcagctggggacatgtctattcgcactggcgctttctctacgagccccagttgccaaatgactacatcggctaaagagagctggcagcccagacagagttgaggccagagcagcttcaaagatgtcttggtgcctgtttcctgtgtgcatgtcagtctcctctgggtaaggcccacatgtgtgtgctcagcaagtctgtatttccttgaccctgagccttctgaccgtacctacatacccaaccgcacctatatacccgaccgcaggttcaactgctgacatcatatgggtcccagtagtgggtacttttgagtgctggtggaatgttatgtgttatgtgtcagtgtgcatttatgtggcaagaagcttgccagtgcggcaggcatttcctgagaagagccatgagaccctgcatgctgcctgaccctggcagtaccacccagaacactttatttgggtgagcctagaccttctgtccacttgagagacaatgacacagctgatctttggaggcttcttgctgtgacctctgatctggctggaagacatgactgctaccctatgccttctgctactcagggtagctctgacatgcttggtgggctccctgggacaaaatactgcctggaccccaagcttactaaagaatccaccctctccaagtctgaggtttccatggaaaccctacactcccacctcactatcccactgacccttcagacagaactaggctagccaaccagaagtctaagactggaacattcaggtcaggcctggaacatcttgaacaggagtgggaaggtagagacatcttcggggaaaatatcccaaagtctcaaaggacagaatagtagcctccagaccctaggttcagttatgctgaaggaagagccctgcttgttggaggttacttattcacaacctacaagaagctacaagctcctaggtagggggaactgcttacgatattctgccctggaatgggtctgagagcacattcctctccagggggttcagaaaagatgtcagaaagggtgtacaggctccttcctcacagctctttgttcttctgcatttcagaggtccccaatggg

SEQ ID NO: 10 - DNA, 1733 bp, Artificial Sequence
  3′ homologous arm
ctcgtggtaacaggtgaggctagtagaacctacgtgggcaattccttcctgcccagagacctcttaggctctctgccatggctctgcctagagccttgacgacactgcccctctccctgtggaaatcctcagatgcccatttacctttaagggatggaagggcttgccaaagtagggtgggtggccagtcactgcccatctaaaatagtcccttgggacttggtgaggacagggtgtgtgaccctaaagaaaatacactatcggtgtcctagaactctattctttgtcatcctgtagagagaatcctggagacctcaacaagatatcccagcccctcgcccaaaccagaaggccggtttcaaggcatggtcattggtatcatgagtgccctagtgggtatccctgtattgctgctgctggcctgggccctagctgtcttctgctcaacaagtatgtcaggtaaggctcatcataccctgcttctgtcctgccaaaccttgtagtcactgtacttcacacatacgtagatcaccagaagggtggtcatgcaccacacacactctgaccactacaaaagcctgtggccgccccacccacacctagcctcaggctgctggctttcctaaacaactagtgagagctgccacctccaggaggtctggtcatcagccagctaagaggccacagctaatatctgctacatgcctaccctgtgttgtggtacaccaggaaaggggacactgatgcacctgtgcctgtggcaggccctactcctcaattcattgtcctaccaggaactccccgttagtaaatgggaagggtgcccgtggggatggaaaggctggtgcttgcccatggtgtagatctcttcagtgcctgacacgcccctcctgagcacacaaaacacacacacacacacacacacacacacacacacacacacacacgagagagaaagatggagagacagagggaggacattcctccactagggaagatggctctgtagctgccctctaacccaaactgtgtgtctcaacagaggccagaggagctggaagcaaggacgacactctggtgagtatgagttttctttcttgagtgatctatcccaggccacccccaggtcttggtacaggtagagagaccatggggcctacagggctagagcctggagagcccagctcccattttctaccaggcccccagagccatatcctgttgttcctcccagcagctgaccccactgtgtgtacccctgtcgtgtccaacgtggtcacgacttgttttcttctgtgcagagacaaggggcaaaagtcaaattttggaatcctaaacccgccaggaaacatttaacgatagaaactgggccagaaacacgaggctgcaccctaaatatcaagaagtcaatggggagcctatggcctctgtgggttctgtgcctgggcagctgttaggtcaggtcccagcttccatgactgaggtgaatttgctctaagaagaaccccaaatccagtgtcagtctggaaacccagcatagggaagggttgagattatgggatgcacacaccaccccccaactgactataacaatggctctttcttctcccccctcccctgccccttgaagaaggaggagccttcagcagcacctgtccctagtgtggcctatgaggagctggacttccagggacgagagaagacaccagagctccctaccgcctgtgtgcaca

SEQ ID NO: 11 - DNA, 333 bp, Artificial Sequence
  canine PD-1 gene fragment
ccctggagcccgctcaccttctccccggcgcagctcacggtgcaggagggagagaacgccacgttcacctgcagcctggccgacatccccgacagcttcgtgctcaactggtaccgcctgagcccccgcaaccagacggacaagctggccgccttccaggaggaccgcatcgagccgggccgggacaggcgcttccgcgtcacgcggctgcccaacgggcgggacttccacatgagcatcgtcgctgcgcgcctcaacgacagcggcatctacctgtgcggggccatctacctgccccccaacacacagatcaacgagagtccccgcgcagag

SEQ ID NO: 12 - DNA, 52 bp, Artificial Sequence
  primer
tttaagaaggagatatacatggctcgagtggcccatagagaccaatgtggac

SEQ ID NO: 13 - DNA, 40 bp, Artificial Sequence
  primer
gagcgggctccagggcccattggggacctctgaaatgcag

SEQ ID NO: 14 - DNA, 25 bp, Artificial Sequence
  primer
gggaaggtagagacatcttcgggga

SEQ ID NO: 15 - DNA, 25 bp, Artificial Sequence
  primer
cgaggggctgggatatcttgttgag

SEQ ID NO: 16 - DNA, 41 bp, Artificial Sequence
  primer
agtccccgcgcagagctcgtggtaacaggtgaggctagtag

SEQ ID NO: 17 - DNA, 42 bp, Artificial Sequence
  primer
ttgttagcagccggatctcagtctagatgtgcacacaggcgg

SEQ ID NO: 18 - DNA, 23 bp, Artificial Sequence
  primer
agggacctccagggcccattggg

SEQ ID NO: 19 - DNA, 23 bp, Artificial Sequence
  primer
cagaggtccccaatgggccctgg

SEQ ID NO: 20 - DNA, 23 bp, Artificial Sequence
  primer
gtagaaggtgagggacctccagg

SEQ ID NO: 21 - DNA, 23 bp, Artificial Sequence
  primer
ccctcaccttctacccagcctgg

SEQ ID NO: 22 - DNA, 23 bp, Artificial Sequence
  primer
gcaccccaaggcaaaaatcgagg

SEQ ID NO: 23 - DNA, 23 bp, Artificial Sequence
  primer
ggagcagagctcgtggtaacagg

SEQ ID NO: 24 - DNA, 23 bp, Artificial Sequence
  primer
gttaccacgagctctgctccagg

SEQ ID NO: 25 - DNA, 23 bp, Artificial Sequence
  primer
gcaaaaatcgaggagagccctgg

SEQ ID NO: 26 - DNA, 19 bp, Artificial Sequence
  sgRNA
tagaaggtgagggacctcc

SEQ ID NO: 27 - DNA, 23 bp, Artificial Sequence
  sgRNA
taggtagaaggtgagggacctcc

SEQ ID NO: 28 - DNA, 19 bp, Artificial Sequence
  sgRNA
ggaggtccctcaccttcta

SEQ ID NO: 29 - DNA, 23 bp, Artificial Sequence
  sgRNA
aaacggaggtccctcaccttcta

SEQ ID NO: 30 - DNA, 19 bp, Artificial Sequence
  sgRNA
caaaaatcgaggagagccc

SEQ ID NO: 31 - DNA, 23 bp, Artificial Sequence
  sgRNA
taggcaaaaatcgaggagagccc

SEQ ID NO: 32 - DNA, 19 bp, Artificial Sequence
  sgRNA
gggctctcctcgatttttg

SEQ ID NO: 33 - DNA, 23 bp, Artificial Sequence
  sgRNA
aaacgggctctcctcgatttttg

SEQ ID NO: 34 - DNA, 132 bp, Artificial Sequence
  sgRNA scaffold
gaattctaatacgactcactatagggggtcttcgagaagacctgttttagagctagaaatagcaagttaaaataaggctagtccgttatcaacttgaaaaagtggcaccgagtcggtgcttttaaaggatcc

SEQ ID NO: 35 - DNA, 25 bp, Artificial Sequence
  primer
catcatactggcaacccctagcctg

SEQ ID NO: 36 - DNA, 24 bp, Artificial Sequence
  primer
gctgtcgttgaggcgcgcagcgac

SEQ ID NO: 37 - DNA, 25 bp, Artificial Sequence
  primer
ctggccgacatccccgacagcttcg

SEQ ID NO: 38 - DNA, 25 bp, Artificial Sequence
  primer
tgacaataggaaaccgggaagcctg